(19) United States
(12) Patent Application Publication
(10) Pub. No.: US 2024/0374569 A1
(43) Pub. Date: Nov. 14, 2024

(54) HDAC3 INHIBITORS FOR THE TREATMENT OF LANGERHANS CELL HISTIOCYTOSIS AND LANGERHANS CELL SARCOMA

(71) Applicant: HENRY FORD HEALTH SYSTEM, DETROIT, MI (US)

(72) Inventors: Qing-Sheng MI, Bloomfield Hills, MI (US); Li ZHOU, Bloomfield Hills, MI (US)

(21) Appl. No.: 18/692,809

(22) PCT Filed: Sep. 22, 2022
(86) PCT No.: PCT/US2022/076857

§ 371 Date: Mar. 16, 2024

Related U.S. Application Data
(60) Provisional application No. 63/246,962, filed on Sep. 22, 2021.

(51) Int. Cl. A61K 31/415; A61P 35/00

(52) U.S. Cl. CPC A61K 31/415; A61P 35/00

(57) ABSTRACT

The present disclosure provides methods for the prevention and treatment of Langerhans cell sarcoma (LCS) and Langerhans cell histiocytosis (LCH) in a subject comprising administering an effective or therapeutically effective dose of a HDAC3 inhibitor to the subject in need thereof.

### CROSS REFERENCE TO RELATED APPLICATIONS

This application claims the benefit of and priority to U.S. Provisional Patent Application No. 63/246,962 filed Sep. 22, 2021, the contents of which are incorporated herein by referenced in its entirety.

### GOVERNMENT SUPPORT

This invention was made with government support under Grant No. AR069681 awarded by the National Institutes of Health (NIH). The Government has certain rights in the invention.

### SEQUENCE LISTING

The instant application contains a Sequence Listing which is submitted herewith in electronically readable XML format via EFS-Web, and is hereby incorporated by reference in its entirety. The electronic Sequence Listing file is filed electronically on Sep. 22, 2022 and is named “25824-517243_Sequence-listing.XML” and is 100,052 bytes in size.

### FIELD

The present disclosure relates to the use of selective HDAC3 inhibitors for the treatment Langerhans cell sarcoma and Langerhans cell histiocytosis.

### BACKGROUND

Dendritic cells (DC) are a heterogeneous group of professional antigen presenting cells (APC) bridging innate and adaptive immunity. Being the sole DC subtype residing at the epidermis, Langerhans cells (LC) constitute 1-2% of epidermal cells and characteristically express C-type lectin langerin, which composes Birbeck granules, the hallmark organelle of LCs. Located in the skin, the body's first line of defense, immature epidermal LCs constantly capture external and internal antigens and migrate to nearby draining lymph nodes (LNs), where they complete maturation process and present antigens to naïve T cells. These mature LCs also secrete cytokines and provide costimulatory signals to influence the decision of T cell responses between immunity and tolerance. As potent immunoregulatory cells, LCs participate in the pathogenesis of various skin disorders, including infection, allergy, autoimmunity and cancer.

Unlike conventional DCs (cDC), epidermal LCs display a unique pattern of ontogeny as well as maintenance. cDCs originate from hematopoietic stem cells within the bone marrow (BM) and are rapidly regenerated by their BM precursors in the adult. By contrast, LCs are derived from embryonic yolk sac erythro-myeloid progenitors and can self-maintain by local proliferation independent of adult hematopoiesis during steady state. However, when local inflammation causes a loss of LCs, the LC compartment is reconstituted by “short-term” LCs, which develop from peripheral blood Gr-1^{hi} monocytes and “long-term” LCs derived from BM. Notably, the ensemble of transcription factors that govern LC homeostasis is different from that for cDCs, although detailed mechanism still remains elusive.

Histone acetylation is an essential post-translational modification of genetic programming, and it is reciprocally regulated by histone acetyltransferases and histone deacetylases (HDACs). HDACs have long been viewed as transcriptional corepressors that regulate chromatin remodeling and gene transcription. Nonetheless, recent studies have shown that HDACs also function as coactivators that deacelylate histone proteins in chromatin to activate gene transcription. Accordingly, the inhibition of HDACs can lead to prominent anti-cancer effects. Accumulating evidence shows that HDAC inhibitors (HDACi) not only dysregulates cDC-mediated T helper cell activation but also suppresses in vitro cDC differentiation from the BM and from the monocyte stages. However, the specific role of individual HDACs in the development, homeostasis and function of epidermal LCs remains unexplored.

The classical HDAC family has 11 members and is classified into three groups: class I RPD3-like proteins (HDAC 1, 2, 3, and 8), class II HDA1-like proteins (HDAC 4, 5, 6, 7, 9, and 10), and a solitary class IV HDAC (HDAC11). HDAC3 is a member of the class I HDACs, and its four different splicing variants have been identified as HD3α, -β, -γ, and -δ. HDAC3 is distinctive within the HDAC family because it is expressed not only in the nucleus but also in the cytoplasm and on the plasma membrane. HDAC3 is also a component of the nuclear receptor co-repressor complex and so has distinct molecular and physiological functions.

HDAC3 is a class I member of HDAC superfamily and plays a critical role in many biological processes, including circadian rhythm of hepatic lipogenesis, intestinal homeostasis maintenance, hematopoietic stem/progenitor DNA replication, brown adipocyte tissue thermogenesis and oocyte maturation. HDAC3 also controls the differentiation of several cell lineages, including regulatory T cells, endothelial cell and osteoblasts.

In addition, HDACs play an important role in cell proliferation and differentiation. HDAC inhibition has been demonstrated modulating the balance between pro- and anti-apoptotic proteins, upregulating the intrinsic and extrinsic apoptosis pathway, and leading to cell cycle arrest. Thus, HDACs are a compelling therapeutic target for cancer therapy. Some HDAC inhibitors have been shown to induce differentiation and cell death in myeloid and lymphoid model systems (Melnick et al., Current Opinion in Hematology 2002, 9, 322).

Langerhans cell histiocytosis (LCH) is the most common histiocytic disorder and standard-of-care treatment remains suboptimal. Histiocytic disorders are a family of diseases most commonly due to persistent mitogen-activated protein kinase (MAPK) signaling in mononuclear phagocytes (MNPs) and their precursors. Within this family, LCH is the most common and predominantly affects children with a similar incidence to childhood Hodgkin lymphoma or acute myeloid leukemia (2.6 to 8.9 cases per million individuals per year). The presence of oncogenic drivers, resistance to apoptosis, self-sufficiency in growth signals, and the ability to invade tissues designated LCH as a type of cancer, although recent data and an evolving perspective on the disease suggests ‘inflammatory myeloid neoplasm’ is a more accurate term. LCH lesions can develop within virtually any organ, leading to a wide range of severity and comorbidities. Current standard of care employs a combination of chemotherapeutic and steroidal anti-inflammatory drugs, which exhibit significant toxic side-effects1. MEK-inhibitors (MEKi), which block MAPK signaling, have shown promise in clinical trials, but they too exhibit toxicity and cessation of MEKi leads to a high rate of LCH recurrence (only ˜50% of patients have progression-free survival). Patients who do not respond to treatment may have lifelong complications such as neurodegenerative LCH (LCH-ND), and rescue therapy remains suboptimal.

### SUMMARY

Langerhans cell sarcoma (LCS) is a neoplastic proliferation of Langerhans cells with notably malignant cytological features, and Langerhans cell histiocytosis (LCH) is a disease characterized by clonal expansion of myeloid precursors that differentiate into CD1a/CD207 in lesions. In a first aspect, the present disclosure provides methods for the prevention, therapeutic treatment, and amelioration of symptoms associated with Langerhans cell histiocytosis (LCH) and Langerhans cell sarcoma (LCS) by administering one or more s HDAC3 inhibitors to a subject in need thereof. In various embodiments, the HDAC3 inhibitor is a selective HDAC3 inhibitor.

In various embodiments, the present disclosure provides methods for the treatment of LCH and LCS in the absence of any other active agents. In some preferred embodiments, LCH and LCS is treated with the selective HDAC3 inhibitors described herein and one or more adjunct or secondary active agents, for example, a chemotherapy agent, an immunotherapy agent, or a MAPK inhibitor, for example, dabrafenib, trametinib, tovorafenib, vemurafenib, GSK2118436 (GSK436) or combinations thereof, wherein the HDAC3 inhibitor and the one or more secondary active agents are administered to the subject in need thereof, concomitantly or separately.

In a third aspect the present disclosure provides a use of a HDAC3 inhibitor for the manufacture of a medicament for preventing or treating LCH or LCS in a subject, comprising administering an effective or therapeutically effective dose to the subject that results in the prevention or treatment of LCH or LCS.

In a fourth aspect, the present disclosure provides a use of a HDAC3 inhibitor for the prevention or treatment of LCH or LCS, comprising administering an effective or therapeutically effective dose to the subject that results in the prevention or treatment of LCH or LCS.

### BRIEF DESCRIPTION OF THE DRAWINGS

The foregoing and other objects, features, and advantages will be apparent from the following description of particular embodiments of the present disclosure, as illustrated in the accompanying drawings. The drawings are not necessarily to scale; emphasis instead being placed upon illustrating the principles of various embodiments of the present disclosure.

FIG. 1A shows HDAC3 mRNA expression levels determined by aRT-PCR in skin fetal macrophages at E14.5 and E16.5 and LC precursors at E18.5 and adult LCs from C57BL/6 mice.

FIG. 1B shows qRT-PCR amplification of HDAC3 mRNA expression in LC precursors from Hdac3fl/fl and Csf1ricreHdac3fl/fl newborn mice (P0).

FIG. 1C shows flow cytometry plot of HDAC3 protein expression in LC precursors from Hdac3fl/fl and Csf1ricreHdac3fl/fl newborns.

FIG. 1D shows flow cytometry data plot of CD11b and F4/80 expression on CD45+ live cells from the fetal skin tissues (E10.5-E16.5) or total live cells from epidermal tissues (E18.5 and P0) of Hdac3fl/fl and Csf1ricreHdac3fl/fl mice.

FIG. 1E shows frequencies of skin fetal macrophages (MF, F4/80hiCD11blo) from the flow cytometry plots in FIG. 1D. Each dot represents one embryo or newborn and bars represent mean±SD from 2-3 independent litters.

FIG. 1F shows frequencies of skin fetal monocytes (MO, F4/80lo CD11bhi) from the flow cytometry plots in FIG. 1D. Each dot represents one embryo or newborn and bars represent mean±SD from 2-3 independent litters.

FIG. 1G shows frequencies of LC precursors (F4/80+CD11b+) in embryonic day 18 and newborn epidermis from the flow cytometry plots in FIG. 1D. Each dot represents one embryo or newborn and bars represent mean±SD from 2-3 independent litters.

FIG. 2A shows the HDAC3 protein expression in LC precursors from Hdac3fl/fl and Csf1ricreHdac3fl/fl newborns (P0) analyzed by flow cytometry. Bar columns showing the frequency of HDAC3+ LCs in the two groups. One dot presents one mouse and data are shown as mean±SD from 2 independent experiments.

FIG. 2B shows F4/80+CD11b+LC precursors sorted from the fetal epidermis of Hdac3fl/fl and Csf1ricreHdac3fl/fl embryos at E18.5 for bulk RNA-seq. Column graphs showing reads per kilobase/per million (RPKM) of the indicated genes from bulk RNA-seq data. Data are shown as mean±SD from 3 independent samples per group. *false discovery rate (FDR)<0.05, **FDR<0.001.

FIG. 3A shows a heat map showing differential gene expression (FDR<0.05) from bulk RNA seq. of F4/80+CD11b+LC precursors sorted from the fetal epidermis of Hdac3fl/fl and Csf1ricreHdac3fl/fl embryos at E18.5.

FIG. 3B shows gene ontology (GO) biological process enrichment of the down-regulated genes in FIG. 3A.

FIG. 3C shows gene set enrichment analysis (GSEA) of the down-regulated genes in FIG. 3A. NES is the normalized enrichment score.

FIG. 3D shows a qRT-PCR analysis of mRNA expression levels of the indicated genes associated with cell cycle and division (n=3/group).

FIG. 3E shows flow cytometry data plot of Ki-67 staining of skin TRMs at E16.5 and epidermal LC precursors at E18.5 from Hdac3fl/fl and Csf1ricreHdac3fl/fl embryos.

FIG. 3F shows a graph plotting the frequency of Ki-67+ tissue resident macrophages in embryonic skin from Hdac3fl/fl and Csf1ricreHdac3 fl/fl embryos.

FIG. 3G shows a graph plotting the frequency of Ki-67+LC precursors in embryonic skin from Hdac3fl/fl and Csf1ricreHdac3 fl/fl embryos.

FIG. 3H shows a Venn diagram of genes identified by HDAC3 ChIP-seq in the LCs from Hdac3fl/fl adult mice overlapped with differentially expressed genes from bulk RNA-seq.

FIG. 3I shows two donut charts representing the number of peaks associated with genes downregulated (left side) and upregulated (right side) in the Csf1ricreHdac3fl/fl mice.

FIG. 3J shows ChIP-qPCR data representing HDAC3 binding at the enhancers of the indicated genes in XS-106 cells. Representative data from two independent experiments.

FIG. 3K shows an ingenuity pathway analysis demonstrating the interactions among HDAC3 and cell cycle genes, which are directly and indirectly regulated by HDAC3. Bars represent mean±SEM from 2-3 independent experiments. N.D., not detectable.

FIG. 4A shows flow cytometry data plot from annexin V+ stained skin resident macrophages (MF) at E16.5 and epidermal LC precursors at E18.5 from Hdac3fl/fl and Csf1ricre Hdac3fl/fl embryos. Bar columns show the frequency of annexin V+ MFs (E16.5) and annexin V+ LCs (E18.5) in the two groups.

FIG. 4B shows the frequency of annexin V+ stained resident macrophage in skin from Hdac3fl/fl and Csf1ricreHdac3fl/fl embryos. One dot represents one mouse, and data are shown as mean±SEM from 2 independent experiments.

FIG. 4C shows the frequency of annexin V+ stained LC precursor cells in Hdac3fl/fl and Csf1ricreHdac3fl/fl embryos. One dot represents one mouse, and data are shown as mean SEM from 2 independent experiments.

FIG. 5A shows HDAC3 mRNA expression from qRT-PCR of LCs isolated from Hdac3fl/fl and huLangerinCreHdac3fl/fl adult mice (n=3/group).

FIG. 5B shows a flow cytometry analysis of HDAC3 protein levels in LCs and keratinocytes (KC) from Hdac3fl/fl (WT) and huLangerinCreHdac3fl/fl (KO) 3-week old mice.

FIG. 5C shows double immunofluorescence staining with Alexa Fluor 488-langerin and PE-MHC-II antibodies of ear epidermal sheets from Hdac3fl/fl and huLangerinCreHdac3 fl/fl mice (n=3 mice analyzed). Images taken on a LEICA DMIRB microscope (Leica Microsystems) at 20× magnification. Scale bars, 50 μm.

FIG. 5D shows a representative flow cytometric analysis of epidermal LCs from Hdac3fl/fl (n=6) and huLangerinCreHDAC3fl/fl (n=5) mice.

FIG. 5E shows a frequency plot of epidermal LCs in Hdac3fl/fl (n=6) and huLangerinCreHDAC3 fl/fl (n=5) mice from the flow cytometry data in FIG. 5D.

FIG. 5F shows the data from a flow cytometric analyses of the percentages of LCs in epidermal cell suspensions at different time points after birth from Hdac3fl/fl (Day 0, n=5; Day 3-6, n=7; Day 10-12, n=13; adult, n=3) and Cd11cCreHdac3fl/fl (Day 0, n=1; Day 3-6, n=3; Day 10-12, n=11; adult, n=3) mice.

FIG. 5G shows LC frequency from a flow cytometry analyses of epidermal cell suspensions at different time points after birth from Hdac3fl/fl (Day 0, n=5; Day 3-6, n=7; Day 10-12, n=13; adult, n=3) and huLangerinCreHDAC3fl/fl (Day 8, n=2; Day 12, n=6; and adult, n=3) mice.

FIG. 5H shows a representative flow cytometry analysis of epidermal Ki-67+MHC-II LCs isolated on day 6 after birth from Hdac3fl/fl (n=5/group) and Cd11cCreHdac3fl/fl (n=3/group) mice.

FIG. 5I shows a frequency plot of Ki-67+ LCs isolated on day 6 after birth from Hdac3fl/fl (n=5/group) and Cd11cCreHdac3fl/fl mice (n=3/group).

FIG. 5J shows a representative flow cytometry analysis of annexin V+ labeled epidermal MHC-II LCs isolated on day 6 after birth from Cd11cCreHdac3fl/fl (KO, n=5) mice and Hdac3fl/fl (Hdac3fl/fl, n=3) littermates.

FIG. 5K shows a frequency data plot of annexin V+ LCs on day 6 after birth from flow cytometry analysis in 5J; Cd11cCreHdac3fl/fl (KO, n=3) mice and Hdac3fl/fl (Hdac3fl/fl, n=5) littermates.

FIG. 5L shows a representative flow cytometry analysis of epidermal Ki-67+MHC-II LCs isolated on day 10 after birth from Hdac3fl/fl (n=4/group) and Cd11cCreHdac3fl/fl mice (n=4/group).

FIG. 5M shows a frequency plot of Ki-67+ LCs isolated on day 10 after birth from Hdac3fl/fl (n=4/group) and Cd11cCreHdac3fl/fl mice (n=4/group).

FIG. 5N shows a representative flow cytometry analysis of annexin V+ labeled epidermal Ki-67+ LCs isolated on day 10 after birth from Hdac3fl/fl (n=4/group) and Cd11cCreHdac3 fl/fl mice (n=4/group).

FIG. 5O shows a frequency plot of an annexin V+ labeled LCs isolated on day 10 after birth from Hdac3fl/fl (n=4/group) and Cd11cCreHdac3 fl/fl mice (n=4/group).

FIG. 5P shows mRNA expression data from qRT-PCR of LC-related genes in LCs from Hdac3fl/fl and huLangerinCreERHdac3 fl/fl mice on day 10 after TAM treatment (n=3/group). HDAC3 expression is normalized to GAPDH.

FIG. 5Q shows Hdac3 mRNA by qRT-PCR from immature LCs (MHC II+CD45+) sorted from freshly isolated epidermal cell suspensions from pooled WT adult mice. Mature LCs were sorted from epidermal cells cultured ex vivo for 60 hrs from pooled WT adult mice. Relative expression levels of HDAC3 are normalized to j-actin in the immature and mature LCs.

FIG. 5R shows a representative flow cytometry analysis of epidermal LCs (MHC II+CD45+) from Hdac3fl/fl (n=10/group) and huLangerinCreERHdac3 fl/fl mice (n=10/group) on day 8-10 after tamoxifen (TAM) injection.

FIG. 5S depicts the LCs (MHC II+CD45+) frequency data from the flow cytometry analysis (as shown in FIG. 5R) from Hdac3fl/fl (n=10/group) and huLangerinCreERHdac3 fl/fl mice (n=10/group) on day 8-10 after tamoxifen (TAM) injection.

FIG. 5T depicts the total number of LCs (MHC II+CD45+) sorted in the flow cytometry analysis (as shown in FIG. 5R) from Hdac3fl/fl (n=10/group) and huLangerinCreERHdac3 fl/fl mice (n=10/group) on day 8-10 after tamoxifen (TAM) injection.

FIG. 5U shows relative levels of HDAC3 mRNA expression by qRT-PCR in the freshly isolated epidermal LCs from Hdac3fl/fl and Hdac3fl/fl huLangerinCreER mice on day 10 after TAM injection. HDAC3 expression level is normalized to GAPDH. Bars show mean±SD and each dot represents one biologically independent sample from pooled mice (n=2-3 mice per sample).

FIG. 5V shows ChIP-qPCR data representing HDAC3 binding at the enhancer regions of Id2 and Runx3 genes in XS-106 cells. Representative data from two independent experiments. Bars represent mean±SEM from 2-3 independent experiments.

FIG. 6A shows a schematic of the experimental plan for producing skin inflammation by UV radiation to examine the effects of HDAC3 deficiency during LC repopulation.

FIG. 6B shows the flow cytometric analysis of the LC population (MHC II+) frequencies on day 0 (untreated) as well as 7 and 21 days after UV treatment (upper panels). The proportions of short-term LCs (MHC II±langerinlow) and long-term LCs (MHC II±langerin±) were determined for each time point (lower panels);

FIG. 6C shows a bar graph representing the frequencies of MHC II±cells from Hdac3fl/fl and Cd11cCreHdac3fl/fl mice at different time points (Day 0, n=3/group; Day 7, n=5/group; Day 21, n=5 Hdac3fl/fl and n=4 Cd11cCreHdac3fl/fl mice).

FIG. 6D shows column graphs representing the frequencies of short-term LCs (MHC II±langerinlow) and long-term (MHC II±langerin±) cells from each genotype at different time points (Day 7, n=4 Hdac3fl/fl and n=5 Cd11cCreHdac3fl/fl mice for both short-term and long-term LCs; Day 21, n=5 Hdac3fl/fl and n=3 Cd11cCreHdac3fl/fl mice for both short-term and long-term LCs).

FIG. 6E shows a schematic of the experimental plan to examine LC repopulation during the inflammatory condition produced by UV radiation; data shown in FIG. 6F-6H.

FIG. 6F shows the flow cytometric analysis of LC population (MHC II±) frequencies on 13 days, 3 weeks and 8 weeks after 30 minutes of UV treatment (upper panels). The proportions of short-term LCs (MHC II±langerinlow) and long-term LCs (MHC II±langerin±) of donor origins were determined for each time point (lower panels).

FIG. 6G shows a bar graph representing the frequencies of donor MHC II±cells from Hdac3fl/fl and Cd11cCreHdac3fl/fl mice at different time points after 30 minutes of UV exposure (Day (D) 13, n=5/group; week (W) 3, n=3/group; W8, n=3/group).

FIG. 6H shows a bar graph representing the frequencies of donor short-term LCs (MHC II±langerinlow) and long-term (MHC II±langerin±) LCs from each genotype at different time points after 30 minutes of UV exposure as in FIG. 9G.

FIG. 6I shows a flow cytometric analysis of the LCs (MHC II±CD45±) frequencies from Runx3fl/fl and Csf1riCreRunx3fl/fl mice at 2-3 weeks old and 6 months old.

FIG. 6J shows a bar graph representing the frequencies of LCs from each genotype of mice for different age groups (2-3 weeks, n=4 Runx3fl/fl and n=5 Csf1riCreRunx3fl/fl mice; 6 months, n=3/group).

FIG. 6K shows the flow cytometric analysis of LC population (MHC II±CD45±) frequencies 14 days after UV treatment (upper panels) Runx3fl/fl and Cd11cCreRunx3fl/fl mice. The proportions of short-term LCs (MHC II langerinlow) and long-term LCs (MHC II±langerin±) were determined (lower panels).

FIG. 6L shows a bar graph representing the frequencies of LCs (MHC-II+CD45+) from Runx3fl/fl and Cd11cCreRunx3fl/fl mice of UV-treated mice (n=5/group).

FIG. 6M shows a bar graph representing the frequencies of short-term LCs (MHC II+langerinlow) and long-term (MHC II+langerin+) cells from flow cytometry analysis in FIG. 6K. Bars represent mean±SEM from 2-3 independent experiments.

FIG. 7A shows a representative flow cytometry analysis of LN migrated LCs (CD207+CD11c+) from Hdac3fl/fl (WT) and huLangerinCreERHdac3fl/fl (KO) mice on day 10 after TAM injection when incubated at 4° C. and 37° C.

FIG. 7B shows a frequency plot of Langerin+MHC-II LCs from the flow cytometry analysis in FIG. 7A.

FIG. 7C shows representative flow cytometric analysis of epidermal LCs (MHC II+Langerin+) and mean fluorescence intensity (MFI) of langerin expression of LCs (n=4/group) derived from freshly isolated epidermal cells from WT and KO mice after incubation at 37° C. or 4° C. (as control) with FITC-Dextran for 45 minutes.

FIG. 7D shows a frequency plot of Langerin-Dextran+ LCs from the flow cytometry analysis in FIG. 7C.

FIG. 7E shows a representative flow cytometric analysis of Langerin expression and CD11c expression in Hdac3fl/fl and huLangerinCreERHdac3fl/fl after TAM treatment.

FIG. 7F shows the relative numbers of LCs in the epidermis (solid line) or skin-draining LNs (dashed line) of huLangerinCreERHdac3fl/fl mice at the indicated number of days after TAM treatment, as a percentage compared with control mice (day 10, n=9/tissue; day 15, n=4/tissue; day 29, n=3/tissue).

FIG. 7G shows a representative flow cytometric analysis (left panels) and the frequencies (right panel) of CD80+ or CD86+ LCs from freshly isolated epidermal cells from tamoxifen-treated Hdac3fl/fl (WT, n=6) and huLangerinCreERHdac3 fl/fl (KO, n=7) mice.

FIG. 7H shows a frequency plot of CD80+ LCs from WT and KO mice flow cytometry analysis data shown in FIG. 7G.

FIG. 7I shows a frequency plot of CD86+ LCs from the WT and KO mice flow cytometry analysis data shown in FIG. 7G.

FIG. 7J shows a representative flow cytometry analysis of CD80+ or CD86+ LCs from epidermal cell suspensions derived from Hdac3fl/fl and huLangerinCreERHdac3 fl/fl mice trunk skin cultured in RPMI for 66-68 hours.

FIG. 7K shows a frequency plot of CD80+ LCs from the flow cytometry analysis in FIG. 7J; Hdac3fl/fl (WT, n=6) and huLangerinCreERHdac3 fl/fl (KO, n=7) mice.

FIG. 7L shows a frequency plot of CD80+ MFs from the flow cytometry analysis in FIG. 7J; Hdac3fl/fl (WT, n=6) and huLangerinCreERHdac3 fl/fl (KO, n=7) mice.

FIG. 7M shows a frequency plot of CD86+ LCs from the flow cytometry analysis in FIG. 7J; Hdac3fl/fl (WT, n=6) and huLangerinCreERHdac3 fl/fl (KO, n=7) mice.

FIG. 7N shows a frequency plot of CD86+ MFs from the flow cytometry analysis in FIG. 7J; Hdac3fl/fl (WT, n=6) and huLangerinCreERHdac3 fl/fl (KO, n=7) mice.

FIG. 8A shows the BMDC culture method of bone marrow cells isolated from mice carrying the BRAFV600E mutation in myeloid lineage cells.

FIG. 8B shows the flow cytometry analysis of BMDCs from BRAFV600ECD11c and WT mice.

FIG. 8C bar graph shows the frequency of total live DCs (LCH or LCH precursors) from WT mice (left) and BRAFV600E mutant mice (right) treated with vehicle or 0.1 μM and 1.0 μM RGFP966.

FIG. 8D shows the chemical structure of the RGFP966 compound.

### DETAILED DESCRIPTION

The details of one or more embodiments of the present disclosure are set forth in the accompanying description below. Although any materials and methods similar or equivalent to those described herein can be used in the practice or testing of the present disclosure, the preferred materials and methods are now described. Other features, objects and advantages of the present disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which the present disclosure belongs. In the case of conflict, the present description will control.

Langerhans cells (LC) are a unique population of mononuclear phagocytes that are seeded from common macrophage precursors in the skin epidermis before birth. They are highly conserved across vertebrate species and this, with their location at the interface with the environment, suggests the strategic importance of LC as immune sentinels at the skin barrier surface.

It was commonly believed that secondary lymphoid organs were the center of T cell immunity, assuming that instructions given during the priming of naïve T cells by migratory and resident dendritic cells (DC) were sufficient for differentiation and effector function by T cells recruited to peripheral sites of tissue injury. However, the field is beginning to appreciate importance of the tissue environment in regulating effector and regulatory T cell function, and it is clear that antigen-presenting cell-T cell interactions play a key role in T cell survival and function outside lymphoid organs. Despite sharing an origin with other tissue-resident macrophages, differentiation of LC is associated with the acquisition of DC-like functions, namely the ability to migrate to skin-draining lymph nodes (LN) and interact with naïve T cells.

The present disclosure provides methods for the treatment of Langerhans cell sarcoma (LCS) and Langerhans cell histiocytosis (LCH) comprising administering to a subject or patient in need thereof, a therapeutically effective amount of a HDAC3 inhibitor.

Langerhans cell sarcoma (LCS) is a neoplastic proliferation of Langerhans cells with notably malignant cytological features.

Langerhans cell histiocytosis (LCH) is a disease characterized by clonal expansion of myeloid precursors that differentiate into CD1a/CD207 (Langerhans cells) in lesions.

Langerhans cell histiocytosis (LCH) is a disorder that primarily affects children, but is also found in adults of all ages. People with LCH produce too many Langerhans cells or histiocytes, a form of white blood cell found in healthy people that is supposed to protect the body from infection. In people with LCH, these cells multiply excessively and build up in certain areas of the body, causing tumors called granulomas to form. The symptoms of LCH vary from person to person, depending on the areas of the body affected. LCH may be found in many areas of the body, including but not limited to the skin and nails, mouth, bones, lungs, liver, spleen, bone marrow, lymph nodes, pituitary gland, and thyroid gland. When it is found in multiple areas of the body, it is known as multisystem disease. The cause of this disease is unknown, although most data suggest that it is characterized by a hyperproliferative growth of immature Langerhans cells.

Certain symptoms or combination of symptoms may be used to diagnose LCH and/or LCS, which include, but not limited to the following: Skin: (Rashes that may be scaly or waxy, Hair loss, and crusty or oily patches on the scalp (may be misdiagnosed as cradle cap); Bone (Bone pain, lumps or lesions on the skull, upper or lower limbs, hands or feet, ribs, pelvis and spine); Stomach (pain in the abdomen or pelvis, yellow skin, vomiting, diarrhea, blood in stool); Liver/spleen (enlarged liver or belly, abnormal blood tests); Lungs (chest pain, problems breathing or shortness of breath, and cough); Brain or Central Nervous System (headaches, dizziness, seizures, blurred or loss of vision, bulging eyeballs, problems swallowing or speaking); Mouth (mouth ulcers, swollen or bleeding gums, swollen lymph nodes in neck); Ears (loss of hearing, discharge from ear canal, redness, and cysts).

High-risk LCH generally involves multiple organs including the liver, bone marrow or spleen. For children with high-risk LCH, the best treatment is chemotherapy (chemo) that lasts about a year.

In some embodiments, the first-line therapy involves a regiment of chemotherapy. Chemotherapy (“chemo”)—uses powerful medicines to kill cells or stop them from growing (dividing) and making more cells. Combination therapy uses more than one type of chemo at a time.

If Langerhans cell histiocytosis is affecting more than one part of the body, the treatment plan. Will likely involve chemotherapy if medical personnel find the disease on the skin and the liver, or find multiple bone lesions.

The standard treatment for Langerhans cell histiocytosis is the use of a combination of chemotherapy drugs. Because chemotherapy treatment doesn't work for everyone, another first-line therapy includes oral administration of an oral drug (either dabrafenib or trametinib) that blocks certain BRAF gene mutations that is associated with Langerhans cell histiocytosis. This “BRAF gene inhibitor” therapy, has been successful in many pediatric patients. It does not cause the side effects of chemotherapy.

Methods of the present disclosure have been based in part on the finding that HDAC3 serves as a key epigenetic regulator that differentially affects Langerhans Cells (LC) development, homeostasis, repopulation, and disease. HDAC3 could be a therapeutic target in Langerhans cell sarcoma and Langerhans cell Histiocytosis.

HDAC inhibitors have been approved by FDA to treat Cutaneous T-cell lymphoma (CTCL) and myeloma. The present disclosure provides for novel methods using HDAC3 inhibitors for treating Langerhans cell sarcoma and Langerhans cell histiocytosis.

Langerhans cell sarcoma (LCS) is a neoplastic proliferation of Langerhans cells with notably malignant cytological features.

Langerhans cell histiocytosis (LCH) is a disease characterized by clonal expansion of myeloid precursors that differentiate into CD1a/CD207 (Langerhans cells) in lesions.

The present disclosure provides a method for treating, preventing or ameliorating the symptoms of Langerhans cell histiocytosis (LCH) or Langerhans cell sarcoma (LCS) in a subject in need thereof, the method comprising administering a therapeutically effective HDAC3 inhibitor to the subject. In various embodiments, HDAC3 inhibitor is a selective inhibitor that inhibits class I HDAC enzymes in a mammal, for example, a human HDAC1, HDAC2 and HDAC3 HDAC. In various embodiments, the HDAC3 inhibitor is a selective inhibitor that predominantly inhibits HDAC3, relative to other class I HDAC species, and other non-class I HDAC species.

### HDAC3 Inhibitors

The selective HDAC3 inhibitor useful in the methods of the present disclosure is more (e.g., at least 1-fold, at least 2-fold, at least 3-fold, at least 4-fold, at least 5-fold more) active for inhibiting HDAC3 than for inhibiting a histone deacetylase that is not HDAC3. In certain embodiments, the selective HDAC3 inhibitor is more active in an in vitro enzymatic inhibition assay for inhibiting HDAC3 than for inhibiting a histone deacetylase that is not HDAC3.

Examples of HDAC3 inhibitors useful in the present disclosure include: Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, U1F010, Tucidinostat (Chidamide), Citarinostat (ACY-241), Domatinostat (4SC-202), and HPOB. Other HDAC3 inhibitors for example selective HDAC3 inhibitors useful in the present disclosure are known in the art, and may also include those described in following US Patent references: U.S. Pat. Nos. 10,125,131, 9,562,013, 9,139,583, and 9,096,549, and U.S. patent application Ser. No. 16/246,320, filed Jan. 11, 2019, Ser. No. 15/389,838, filed Dec. 23, 2016, Ser. No. 14/753,913, filed Jun. 29, 2015, Ser. No. 14/169,775, filed Jan. 31, 2014, Ser. No. 14/169,732, filed Jan. 31, 2014, the disclosures of all of the preceding patent references are incorporated by reference herein in their entireties.

### HDAC3 Inhibitor Compositions for Use in Treatment and Prevention of LCH and LCS

In another aspect, the invention provides a pharmaceutical composition comprising a selective HDAC3 inhibitor described herein or a pharmaceutically acceptable salt thereof, together with a pharmaceutically acceptable carrier. In various embodiments, HDAC3 inhibitors of the present invention, may be HDAC3 inhibitors that also may inhibit, other HDAC species such as HDACC1, HDAC2 and HDAC8, but in general, are more selective against HDAC3 than other HDAC isoforms. In some embodiments, the present disclosure provides the use of selective HDAC3 inhibitors for the treatment, prevention and amelioration of symptoms associated with LCH and LCS. In one aspect, the invention provides compounds that possess thermodynamic and kinetic binding selectivity for HDAC3 over HDAC1 and HDAC2 and HDACs 4, 5, 6, 7, 8 and 9. While there are HDAC inhibitors in the clinic, most of these do not show significant selectivity for an individual HDAC isoform. Non-selective HDAC inhibitors may be associated with adverse effects. For example, some non-selective inhibitors are believed to be associated with undesirable hematological side effects, such as anemia and thrombocytopenia (loss of platelets). Other adverse side effects related to non-selective HDAC inhibition include fatigue, anorexia, and GI-toxicity. In certain embodiments, the HDAC3 inhibitor reduces the activity of one histone deacetylase (e.g., HDAC3) or a sub-group of histone deacetylases (e.g., HDAC1, HDAC2, and HDAC3) to a greater extent than other histone deacetylases. Where the compound preferentially reduces the activity of a sub-group of histone deacetylases, the reduction in activity of each member of the sub-group may be the same or different.

In certain embodiments, the present invention relates to the aforementioned HDAC3 inhibitor. In one aspect, a HDAC3 inhibitor is a selective HDAC3 inhibitor. In another embodiment, the HDAC3 inhibitor is a HDAC1, 2, 3 selective inhibitor.

In one embodiment, a HDAC3 inhibitor is selective for HDAC3, and will have at least about 1-fold, or 2-fold (e.g., at least about 5-fold, 10-fold, 15-fold, or 20-fold) greater activity to inhibit HDAC3 as compared to one or more other HDACs (e.g., one or more HDACs of class I or II, one or more of HDAC1 and HDAC2).

In one aspect, a compound of the invention possesses thermodynamic and kinetic binding selectivity for HDAC3 over HDAC1 and HDAC2. Long residence times are advantageous in terms of duration of pharmacological effect and target selectivity. Long residence times can also mitigate off-target toxicity (Copeland, R. A. et al., Nature, 5, 730-739 (2006); Copeland, R. A. et al., Future Med. Chem. 3(12), 1491-1501 (2011)).

In one embodiment, a HDAC3 inhibitor, for example, a selective HDAC3 inhibitor selectively inhibits at least one, class I HDAC enzymes with an IC₅₀ value greater than 0.0000001 μM and less than or equal to 0.1 μM, 1 μM, 5 μM, 20 μM, 30 μM, 50 μM, or 75 μM. In another embodiment, a HDAC3 inhibitor selectively inhibits at least two, class I HDAC enzymes with an IC₅₀ value greater than 0.0000001 μM and less than or equal to 0.1 μM, 1 μM, 5 μM, 20 μM, 30 μM, 50 μM, or 75 μM. In one embodiment, a HDAC3 inhibitor, for example, a selective HDAC3 inhibitor selectively inhibits HDAC3 enzyme in a human subject, or equivalent thereof, with an IC₅₀ value greater than 0.0000001 μM and less than or equal to 0.1 μM, 1 μM, 5 μM, 20 μM, 30 μM, 50 μM, or 75 μM. In another embodiment, a compound selectively inhibits an HDAC3 enzyme, with an IC₅₀ value greater than 0.0000001 μM and less than or equal to 0.1 μM, 1 μM, 5 μM, 20 μM, 30 μM, 50 μM, or 75 μM. In one embodiment, the invention provides a method of treating, alleviating, and/or preventing LCH or LCS in a subject, for example a human subject, the method comprising administering to the subject in need thereof an effective amount of a HDAC3 inhibitor, for example, a selective HDAC3 inhibitor of the invention or a pharmaceutically acceptable salt, hydrate, solvate, or prodrug thereof.

The pharmaceutical compositions of the present invention comprise a therapeutically effective amount of a HDAC3 inhibitor, for example, a selective HDAC3 inhibitor of the present invention formulated together with one or more pharmaceutically acceptable carriers. The term “pharmaceutically acceptable carrier” means a non-toxic, inert solid, semi-solid or liquid filler, diluent, encapsulating material or formulation auxiliary of any type. The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

Selective HDAC3 inhibitor compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a selective HDAC3 inhibitor compound of the present invention in free form or in a pharmaceutically acceptable salt form in association with at least one pharmaceutically acceptable excipient, carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The HDAC3 inhibitor, for example, a selective HDAC3 inhibitor compound can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

### Treatment of Langerhans Cell Sarcoma (LCS) and Langerhans Cell Histiocytosis (LCH)

In certain embodiments, the subject is identified as in need of such treatment for Langerhans cell sarcoma (LCS) and Langerhans cell histiocytosis (LCH) is administered a therapeutically effective amount of a HDAC3 inhibitor, for example, a selective HDAC3 inhibitor, or a pharmaceutical composition comprising a HDAC3 inhibitor, for example, a selective HDAC3 inhibitor, to a subject in need thereof, in such amounts and for such time as is necessary to achieve the desired result. In certain embodiments of the present invention, a “therapeutically effective amount” of the selective HDAC3 inhibitor or pharmaceutical composition is that amount effective for reducing or eliminating the growth of the histiocytes, or Langerhans cells involved in LCS or LCH. The HDAC3 inhibitors and compositions, according to the methods of the present disclosure, can be administered using any amount, and any route of administration effective for reducing or eliminating the growth of the histiocytes, or Langerhans cells involved in LCS or LCH. Thus, the expression “amount effective to reduce or eliminate the growth of the histiocytes, or Langerhans cells involved in LCS or LCH” as used herein, refers to a sufficient amount of a HDAC3 inhibitor, for example, a selective HDAC3 inhibitor to inhibit the growth of said histiocytes or Langerhans cells involved in the pathology of LCH and LCS. The exact amount required will vary from subject to subject, depending on the species, age, and general condition of the subject, the severity of the infection, the particular anticancer agent, its mode of administration, and the like.

In some embodiments, the method is a method of inhibiting (e.g., by at least 5%, at least 10%, at least 20%, at least 30%, at least 40%, at least 50%, at least 60%, at least 70%, at least 80%, at least 90%, or about 100%) proliferation of LCS and LCH, or inducing death (e.g., by at least 5%, at least 10%, at least 20%, at least 30%, at least 40%, at least 50%, at least 60%, at least 70%, at least 80%, at least 90%, or about 100%) of LCS or LCH in a subject, the method comprising administering a selective HDAC3 inhibitor as described herein, to a subject in need thereof as described herein, e.g., a subject having LCS or LCH, or predisposed for developing LCH or LCS. In some embodiments, the selective HDAC3 inhibitor and the MAPK inhibitor, and/or immunotherapy and/or chemotherapy agent(s) act synergistically to treat LCH or LCS compared to the HDAC3 inhibitor used alone.

In various embodiments, selective HDAC3 inhibitors are administered in therapeutically effective doses to treat LCS or LCH in a subject in need thereof.

The treatment regimens and pharmaceutical compositions described herein can be administered intravenously, subcutaneously, topically, orally, or by any other route described herein. In one approach, a loading-dose of a HDAC3 inhibitor, for example, a selective HDAC3 inhibitor is administered to a subject in need thereof, followed by maintenance dosing administered orally.

In various embodiments, HDAC3 inhibitors useful in the methods of the present disclosure as provided herein, for use as a single agent or for use in a combination treatment modality, with a MAPK inhibitor, for example, a BRAF inhibitor, an AKT inhibitor or a MEK inhibitor or a chemotherapeutic agent, (for example, including but not limited to cytarabine, vinblastine, cyclophosphamide, thalidomide, and leucovorin) can include one or more of the following HDAC3 inhibitors: Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, U1F010, Tucidinostat (Chidamide), Citarinostat (ACY-241), Domatinostat (4SC-202), and HPOB. Other selective HDAC3 inhibitors useful in the present disclosure are known in the art, and may also include those described in following US Patent references: U.S. Pat. Nos. 10,125,131, 9,562,013, 9,139,583, and 9,096,549, and U.S. patent application Ser. No. 16/246,320, filed Jan. 11, 2019, Ser. No. 15/389,838, filed Dec. 23, 2016, Ser. No. 14/753,913, filed Jun. 29, 2015, Ser. No. 14/169,775, filed Jan. 31, 2014, Ser. No. 14/169,732, filed Jan. 31, 2014, the disclosures of all of the preceding patent references are incorporated by reference herein in their entireties.

In various embodiments, methods for the prevention, treatment or amelioration of symptoms associated with LCH or LCS in a subject, for example a mammal, for example a human subject in need thereof, comprises administering a therapeutically effective dose of a HDAC3 inhibitor to the subject. In related embodiments, the HDAC3 inhibitor is selected from: Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, U1F010, Tucidinostat (Chidamide), Citarinostat (ACY-241), Domatinostat (4SC-202), and HPOB. In related embodiments, the HDAC3 inhibitor is a selective HDAC3 inhibitor. In related embodiments, the selective HDAC3 inhibitor is RGFP966 ((2E)-N-(2-Amino-4-fluorophenyl)-3-[(2E)-1-(3-phenyl-2-propen-1-yl)-1H-pyrazol-4-yl]-2-propenamide, (E)-N-(2-amino-4-fluorophenyl)-3-(1-cinnamyl-1H-pyrazol-4-yl)acrylamide, CAS No. 1396841-57-8), having the structure:

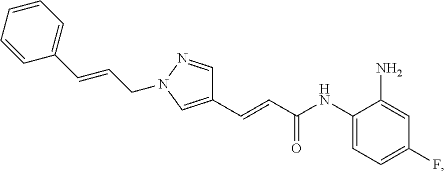 [SMILES: Nc1cc(F)ccc1NC(=O)/C=C/c1cnn(C/C=C/c2ccccc2)c1]

or a pharmaceutically acceptable salt thereof. RGFP966 is commercially available from Selleckchem, Catalog No. S7229; Radnor, PA, USA. In some embodiments, the present disclosure provides the use of a HDAC3 inhibitor for the manufacture of a medicament for preventing or treating LCH or LCS in a subject comprising administering an effective or therapeutically effective dose to the subject that results in the prevention or treatment of LCH or LCS. In other embodiments, the present disclosure provides use of a HDAC3 inhibitor for the prevention or treatment of LCH or LCS, comprising administering an effective or therapeutically effective dose to the subject that results in the prevention or treatment of LCH or LCS. In related embodiments, the HDAC3 inhibitor as mentioned in all of the methods above comprises one or more selective HDAC3 inhibitors. In related embodiments, the HDAC3 inhibitor is selected from: Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, UF010, Tucidinostat (Chidamide), Citarinostat (ACY-241), Domatinostat (4SC-202), and HPOB. In related embodiments, the HDAC3 inhibitor is a selective HDAC3 inhibitor. In related embodiments, the selective HDAC3 inhibitor is RGFP966 ((2E)-N-(2-Amino-4-fluorophenyl)-3-[(2E)-1-(3-phenyl-2-propen-1-yl)-1H-pyrazol-4-yl]-2-propenamide, (E)-N-(2-amino-4-fluorophenyl)-3-(1-cinnamyl-1H-pyrazol-4-yl)acrylamide, CAS No. 1396841-57-8). In another embodiment, illustrative selective HDAC3 inhibitors useful in the present methods and compositions include HDAC3 inhibitors described in Jian Liu, et al., Discovery of Highly Selective and Potent HDAC3 Inhibitors Based on a 2-Substituted Benzamide Zinc Binding Group, ACS Medicinal Chemistry Letters 2020 11 (12), 2476-2483, the disclosure of which is incorporated herein by reference in its entirety.

### Administration, and Dosing Regimen

In general, selective HDAC3 inhibitor compounds of the invention will be administered in therapeutically effective amounts via any of the usual and acceptable modes known in the art, either singly or in combination with one or more therapeutic agents. Pharmaceutical compositions are administered in a manner appropriate to the disease to be treated (or prevented). An appropriate dose and a suitable duration and frequency of administration will be determined by such factors as the condition of the patient, the type and severity of the patient's LCH or LCS disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In general, effective or therapeutically effective doses of any of the HDAC3 inhibitors described herein to be obtained systemically at daily dosages ranging from about 0.01 to 1,000 mg/kg or from about 1 mg/kg to about 200 mg/kg per body weight, for any of the methods described in the present application for the treatment, prevention and amelioration of symptoms associated with LCH or LCS. An indicated daily dosage in an exemplary larger mammal, e.g. humans, is in the range from about 0.5 mg to about 1000 mg per dose, or per daily dose, or per weekly dose, conveniently administered, e.g. in divided doses up from one to four times a day or in retard form. Suitable unit dosage forms for oral administration comprise from ca. 1 to 500 mg of the HDAC3 inhibitor, for example, a selective HDAC3 inhibitor.

In certain embodiments, a therapeutic amount or effective dose, or effective daily dose, or effective weekly dose of the HDAC3 inhibitor, for example, a selective HDAC3 inhibitor of the present disclosure, may range from about 0.001 mg/kg to about 500 mg/kg (about 0.018 mg/m² to about 900 mg/m²), alternatively from about 0.1 mg/kg to about 100 mg/kg (about 0.18 to about 180 mg/m²). In some embodiments, therapeutically effective dose, or therapeutically effective daily dose or therapeutically effective weekly dose, can include an amount ranging from about 10 mg to about 300 mg, dosed, once or more per day, for two or more days per week, for at least one to 30 weeks per regimen. In some embodiments, the HDAC3 inhibitor is dosed to a subject with LCH or LCS in doses ranging from about 10 mg-50 mg per dose, administered per oral, two to three times per week, for a duration ranging from about one day to 20 weeks. In alternate embodiments, the HDAC3 inhibitor is dosed to a subject in need thereof, for use in an intravenous, drip, starting from about 5 mg—to about 300 mg per 10 mL, per 100 ml, or per 1,000 mL IV fluid volume (and all mL intervals therein) for one or more doses, per week, once per week, or one or more doses every two weeks, or one or more doses every three weeks, or one or more doses every four weeks. The regimen just described may incorporate effective doses of a HDAC3 inhibitor, therapeutically effective doses of a HDAC3 inhibitor, or sub-therapeutic effective doses of a HDC3 inhibitor, for example, when the HDAC3 inhibitor is coadministered with a secondary active agent as discussed herein.

In general, treatment regimens according to the present invention comprise administration to a patient in need of such treatment from about 10 mg to about 1000 mg of the HDAC3 inhibitor, for example, a selective HDAC3 inhibitor of this invention per day in single or multiple doses. Therapeutic amounts or doses will also vary depending on route of administration, as well as the possibility of co-usage with other secondary agents.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the selective HDAC3 inhibitor compounds and compositions of the present invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### Combination Treatments

In various embodiments, the present disclosure provides methods for preventing LCH and LCS, or treating LCH and LCS, or ameliorating symptoms associated with LCH and LCS using a combination of a HDAC3 inhibitor, for example, a selective HDAC3 inhibitor and a secondary active agent selected from, a chemotherapeutic agent, an immunomodulating agent, a MAPK inhibitor or combinations thereof.

In some embodiments, the present disclosure provides a method for the prevention of LCH or LCS, treatment of LCH or LCS, or amelioration of symptoms associated with LCH or LCS in a subject, for example a mammal, for example a human subject in need thereof, the method comprises administering a therapeutically effective combination comprising a HDAC3 inhibitor and a secondary active agent, wherein the secondary active agent is a MAPK inhibitor, and administering a therapeutically effective dose of the HDAC3 inhibitor or MAPK inhibitor to the subject to prevent LCH or LCS, treat LCH or LCS, or ameliorate the symptoms associated with LCH or LCS.

As used herein, “MAPK inhibitor” includes any agent which inhibits, downregulates or decreases activity of any component of the MAPK signaling pathway, including BRAF, MEK, ERK, etc. See, Nikiforov, Y., Thyroid carcinoma: molecular pathways and therapeutic targets, Modern Pathology, 2008, 21:S37-43, which is incorporated herein by reference. In some embodiments, the MAPK inhibitor is a BRAF inhibitor. In related embodiments, the BRAF inhibitor inhibits and is effective against BRAFV600 mutations, for example, BRAFV600E mutations.

In another embodiment, the MAPK inhibitor is RAF265 (also called CHIR-265). RAF265 is an orally bioavailable small molecule with preclinical antitumor activity that currently is being tested in clinical trials. Much like sorafenib, in vitro kinase assays show RAF265 inhibits the activities of several intracellular kinases, including BRAF (V600E), BRAF (wild type), c-RAF, VEGF receptor 2 (VEGFR2), platelet-derived growth factor receptor (PDGFR), colony-stimulating factor (CSF)1R, RET and c-KIT, SRC, STE20, and others with IC50 ranging from less than 20 to more than 100 nmol/L. However, in cell-based assays, RAF265 is most potent for BRAFV600E, and VEGFR2, but less active for PDGFRB and c-KIT. See, Su et al, RAF265 Inhibits the Growth of Advanced Human Melanoma Tumors, Clinical Cancer Research, Apr. 15, 2012, 18:2184, which is incorporated herein by reference. RAF265 has a molecular weight of 518.41 and is available from Selleckchem.com.

In one embodiment, the MAPK inhibitor is PLX4720. In another embodiment, the MAPK inhibitor is PLX4032 (vemurafenib). PLX4720 is a structurally closely related precursor of vemurafenib. In another embodiment, the MAPK inhibitor includes PLX4720 and PLX4032. See, Michaelis et al, Differential effects of the oncogenic BRAF inhibitor PLX4032 (vemurafenib) and its progenitor PLX4720 on ABCB1 function, Journal of Pharmacy and Pharmaceutical Sciences, 2014, 17(1):154-68, which is incorporated herein by reference.

In another embodiment, the MAPK inhibitor is AZD6244 (Selumetinib). Selumetinib (AZD6244) is a potent, highly selective MEK1 inhibitor with IC50 of 14 nM in cell-free assays, also inhibits ERK1/2 phosphorylation with IC50 of 10 nM, no inhibition to p38a, MKK6, EGFR, ErbB2, ERK2, B-Raf, etc. Clinical trials of Selumetinib in patients with BRAFv600E/K-mutated melanoma are ongoing (see, e.g., Catalanotti et al, Phase II trial of MEK inhibitor selumetinib (AZD6244, ARRY-142886) in patients with BRAFV600E/K-mutated melanoma, Clinical Cancer Research, 2013 Apr. 15; 19(8):2257-64, which is incorporated by reference). AZD6244 has a molecular weight of 457.68 and is available from Selleckchem.com.

In another embodiment, the MAPK inhibitor is PD0325901. A second-generation oral MEK inhibitor, compound PD 0325901 demonstrates relatively minor changes in the chemical structure of PD 0325901 as compared to its predecessor CI-1040. The cyclopropylmethoxy group of CI-1040 was replaced with a (R)-dihydroxy-propoxy group and the 2-chloro substituent of CI-1040 was replaced with a 2-flouro group on the second aromatic ring, resulting in significant increases in potency. See, e.g., Wang et al, 2007, cited above.

In another embodiment, the MAPK inhibitor is LGX818 (encorafenib). Encorafenib is a novel oral small molecule kinase inhibitor with potent and selective inhibitory activity against mutant BRAF kinase. LGX818 is currently in phase 3 clinical trials for treatment of BRAF V600 mutant melanoma. LGX818 has a molecular weight of 540.01 and is property of Novartis.

In another embodiment, the MAPK inhibitor is MEK162 (binimetinib). MEK162 is a highly selective, orally bioavailable, ATP-uncompetitive inhibitor of MEK1/2. In previous preclinical work, MEK162 was found to be highly effective in inhibiting growth of xenograft tumors regardless of Ras/Raf pathway deregulation. MEK162 is currently in clinical trials for patients with RAS/RAF/MEK activated tumors. See, Ascierto et al, MEK162 for patients with advanced melanoma harboring NRAS or Val600 BRAF mutations (also referred to as BRAFV600E) or: a non-randomized, open-label phase 2 study, Lancet Oncology, 2013 March; 14(3):249-56, which is incorporated herein by reference. MEK162 has a molecular weight of 441.23 and is property of Novartis.

In another embodiment, the MAPK inhibitor is dabrafenib. Dabrafenib acts as an inhibitor of the associated enzyme B-Raf, which plays a role in the regulation of cell growth. Dabrafenib has clinical activity with a manageable safety profile in clinical trials of phase 1 and 2 in patients with BRAF (V600)-mutated metastatic melanoma. Dabrafenib was approved as a single agent treatment for patients with BRAF V600E mutation-positive advanced melanoma on May 30, 2013. Clinical trial data demonstrated that resistance to dabrafinib and trametinib occurs within 6 to 7 months. To overcome this resistance, the BRAF inhibitor dabrafenib was combined with the MEK inhibitor trametinib. On Jan. 8, 2014, the FDA approved the combination of dabrafenib and trametinib for the treatment of patients with BRAF V600E/K-mutant metastatic melanoma. Thus, in on embodiment, the MAPK inhibitor includes dabrafenib and trametinib. In another embodiment, the MAPK inhibitor is trametinib.

In another embodiment, the MAPK inhibitor includes more than one agent. In one embodiment, the MAPK inhibitor includes PLX4720 and PD0325901. In another embodiment, the MAPK inhibitor includes dabrafenib and trametinib. In yet another embodiment, the MAPK inhibitor includes LGX818 and MEK162.

As used herein, “MAPK inhibitor” includes the specific MAPK inhibitor compounds described herein, and salts derived from pharmaceutically or physiologically acceptable acids, bases, alkali metals and alkaline earth metals. Physiologically acceptable acids include those derived from inorganic and organic acids. A number of inorganic acids are known in the art and include, without limitation, hydrochloric, hydrobromic, hydroiodic, sulfuric, nitric, and phosphoric acid. A number of organic acids are also known in the art and include, without limitation, lactic, formic, acetic, fumaric, citric, propionic, oxalic, succinic, glycolic, glucuronic, maleic, furoic, glutamic, benzoic, anthranilic, salicylic, tartaric, malonic, mallic, phenylacetic, mandelic, embonic, methanesulfonic, ethanesulfonic, panthenoic, benzenesulfonic, toluenesulfonic, stearic, sulfanilic, alginic, and galacturonic acids. In another embodiment, the MAPK inhibitor includes any MAPK inhibitor known in the art or developed hereafter. Physiologically acceptable bases include those derived from inorganic and organic bases. A number of inorganic bases are known in the art and include, without limitation, aluminum, calcium, lithium, magnesium, potassium, sodium, and zinc sulfate or phosphate compounds, among others. Organic bases include, without limitation, N,N-dibenzylethylenediamine, chloroprocaine, choline, diethanolamine, ethylenediamine, meglumine, and procaine, among others. Physiologically acceptable alkali salts and alkaline earth metal salts include, without limitation, sodium, potassium, calcium and magnesium salts, optionally in the form of esters, and carbamates. The MAPK inhibitor compound salts can be also in the form of esters, carbamates, sulfates, ethers, oximes, carbonates, and other conventional “pro-drug” forms, which, when administered in such form, convert to the active moiety in vivo. In one embodiment, the prodrugs are esters.

The MAPK inhibitor compounds discussed herein also encompasses “metabolites” which are unique products formed by processing the PI3K inhibitor compound by the cell or subject. In one embodiment, metabolites are formed in vivo.

As used herein, the term “effective amount” or “pharmaceutically effective amount” as it refers to individual composition components, refers to the amount of e.g., the selected MAPK inhibitor described herein that elicits the biological or medicinal response in a tissue, system, animal, individual or human that is being sought by a researcher, veterinarian, medical doctor or other clinician, which includes one or more of the following, preventing a disease; e.g., inhibiting a disease, condition or disorder in an individual that is experiencing or displaying the pathology or symptomatology of the disease, condition or disorder (i.e., arresting or slowing further development of the pathology and/or symptomatology); ameliorating a disease, condition or disorder, i.e. LCH or LCS in an individual that is experiencing or displaying the pathology or symptomatology of LCH or LCS (i.e., reversing the pathology and/or symptomatology); and inhibiting a physiological process. For example, an effective amount of a combination of a HDAC3 inhibitor, for example, a selective HDAC3 inhibitor and a MAPK inhibitor, for example a BRAF inhibitor, when administered to a subject to treat LCH or LCS, is sufficient to inhibit, slow, reduce, or eliminate tumor growth in a subject having LCH or LCS.

The effective dosage or amount of the second active agent of the combination with a HDAC3 inhibitor, i.e. a MAPK inhibitor may vary depending on the particular compound employed, the mode of administration, the type and severity of the condition being treated, and subject being treated as determined by the subject's physician. The effective dosage of each active component (e.g., a HDAC3 inhibitor and a MAPK inhibitor) is generally individually determined, although the dosages of each of the HDAC3 inhibitor and the MAPK inhibitor can be the same. In one embodiment, the dosage of each of the HDAC3 inhibitor and the MAPK inhibitor in the combination is about 0.001 mg to about 1,000 mg. In one embodiment, the effective amount is or about 0.1 to about 500 mg/kg of body weight, or about 0.1 to about 200 mg/kg of body weight, including any intervening amount. In another embodiment, the effective amount of each of the HDAC3 inhibitor and the MAPK inhibitor in the combination, is about 0.5 to about 40 mg/kg. In a further embodiment, the effective amount of the HDAC3 inhibitor and the MAPK inhibitor is about 0.7 to about 30 mg/kg. In still another embodiment, the effective amount of the HDAC3 inhibitor and the MAPK inhibitor is about 1 to about 20 mg/kg. In yet a further embodiment, the effective amount of each of the HDAC3 inhibitor and the MAPK inhibitor independently is about 0.001 mg/kg to about 1000 mg/kg body weight. In another embodiment, the effective amount of each of the HDAC3 inhibitor and the MAPK inhibitor is less than about 5 g/kg, about 500 mg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 0.075 mg/kg, about 0.05 mg/kg, or about 0.01 mg/kg. However, the effective amount of the MAPK inhibitor compound can be determined by the attending physician and depends on the condition treated, the compound administered, the route of delivery, age, weight, and severity of the patient's symptoms and response pattern of the patient.

Toxicity and therapeutic efficacy of the compounds (HDAC3 inhibitor and/or the MAPK inhibitor, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. HDAC3 and MAPK inhibitor compounds which exhibit high therapeutic indices are preferred. While HDAC3 and MAPK inhibitor compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays (such as those described in the examples below) and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

One or more of the combination of HDAC3 and MAPK inhibitor compounds discussed herein may be administered in combination with other pharmaceutical agents, as well as in combination with each other. The term “pharmaceutical” agent as used herein refers to a chemical compound which results in a pharmacological effect in a patient. A “pharmaceutical” agent can include any biological agent, chemical agent, or applied technology which results in a pharmacological effect in the subject.

In addition to the combination of HDAC3 inhibitors with one or more MAPK inhibitor compounds, described above, the HDAC3 inhibitor, for example, a selective HDAC3 inhibitor compound may be combined with a secondary active agent or one or more therapeutic agents which are used to treat solid tumors. In one embodiment, the secondary active agent is a chemotherapeutic. Examples of chemotherapeutic agents include those recited in the “Physician's Desk Reference”, 64^{th} Edition, Thomson Reuters, 2010, which is hereby incorporated by reference. Therapeutically effective amounts of the additional medication(s) or therapeutic agents are well known to those skilled in the art. However, it is well within the attending physician to determine the amount of other medication to be delivered.

In some embodiments, the present disclosure provides a method for the prevention of LCH or LCS, treatment of LCH or LCS, or amelioration of symptoms associated with LCH or LCS in a subject, for example a mammal, for example a human subject in need thereof, the method comprises administering a therapeutically effective combination comprising a HDAC3 inhibitor and one or more secondary active agents, wherein the one or more secondary active agents is one or more chemotherapeutic agents, and administering a therapeutically effective dose of the HDAC3 inhibitor or chemotherapeutic agent(s) to the subject to prevent LCH or LCS, treat LCH or LCS, or ameliorate the symptoms associated with LCH or LCS.

In one embodiment, the chemotherapeutic agent is selected from among cisplatin, carboplatin, 5-fluorouracil, cyclophosphamide, oncovin, vincristine, prednisone, or rituximab, mechlorethamine, cyclophosphamide, ifosfamide, melphalan, chlorambucil, carmustine, lomustine, semustine, thriethylenemelamine, triethylene thiophosphoramide, hexamethylmelamine altretamine, busulfan, triazines dacarbazine, methotrexate, trimetrexate, fluorodeoxyuridine, gemcitabine, cytosine arabinoside, 5-azacytidine, 2,2′-difluorodeoxycytidine, 6-mercaptopurine, 6-thioguanine, azathioprine, 2′-deoxycoformycin, erythrohydroxynonyladenine, fludarabine phosphate, 2-chlorodeoxyadenosine, camptothecin, topotecan, irinotecan, paclitaxel, vinblastine, vincristine, vinorelbine, docetaxel, estramustine, estramustine phosphate, etoposide, teniposide, mitoxantrone, mitotane, aminoglutethimide or combinations thereof.

The effective dosage or amount of the secondary active agent of the combination with a HDAC3 inhibitor, i.e. a Chemotherapeutic may vary depending on the particular compound employed, the mode of administration, the type and severity of the condition being treated, and subject being treated as determined by the subject's physician. The effective dosage of each active component (e.g., a HDAC3 inhibitor and a chemotherapeutic agent) is generally individually determined, although the dosages of each active agent can be the same or different. In one embodiment, the dosage of the chemotherapeutic agent is about 0.001 mg to about 1000 mg. In one embodiment, the effective amount chemotherapeutic agent is or about 0.1 to about 500 mg/kg of body weight, or about 0.1 to about 200 mg/kg of body weight, including any intervening amount. In another embodiment, the effective amount of the chemotherapeutic agent in the combination with an HDAC3 inhibitor, is about 0.1 to about 100 mg/kg. In a further embodiment, the effective amount is about 0.7 to about 75 mg/kg. In still another embodiment, the effective amount is about 1 to about 50 mg/kg. In yet a further embodiment, the effective amount of the chemotherapeutic agent is about 0.001 mg/kg to 1,000 mg/kg body weight. In another embodiment, the effective amount of the chemotherapeutic agent for use in the combination with a HDAC3 inhibitor, is less than about 5 g/kg, about 500 mg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 0.075 mg/kg, about 0.05 mg/kg, or about 0.01 mg/kg. However, the effective amount of the chemotherapeutic agent can be determined by the attending physician and depends on the condition treated, the compound administered, the route of delivery, age, weight, and severity of the patient's symptoms and response pattern of the patient.

Toxicity and therapeutic efficacy of the compounds (HDAC3 inhibitor and/or the chemotherapeutic agent, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. HDAC3 and Chemotherapeutic compounds which exhibit high therapeutic indices are preferred. While HDAC3 and Chemotherapeutic compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

In some exemplary embodiments, illustrative combinations of HDAC3 inhibitors and secondary active agents for the methods for preventing LCH and LCS, or treating LCH and LCS, or ameliorating symptoms associated with LCH and LCS as disclosed herein comprises a HDAC3 inhibitor, for example, a selective HDAC3 inhibitor and a secondary active agent, wherein the secondary agent is a MAPK inhibitor. In a related embodiment, an illustrative embodiment comprises a combination of a HDAC3 inhibitor, for example a selective HDAC3 inhibitor and BRAF mutation inhibitor. In a related embodiment, an illustrative embodiment comprises a combination of a HDAC3 inhibitor, for example a selective HDAC3 inhibitor and a BRAFV600 mutation inhibitor. In a related embodiment, an illustrative embodiment comprises a combination of a HDAC3 inhibitor, for example a selective HDAC3 inhibitor and a BRAFV600E mutation inhibitor. In a related embodiment, an illustrative embodiment comprises a combination of a HDAC3 inhibitor, for example a selective HDAC3 inhibitor and a MEK inhibitor. In a related embodiment, an illustrative embodiment comprises a combination of a HDAC3 inhibitor, for example a selective HDAC3 inhibitor and Akt1 mutation inhibitor. In an illustrative embodiment comprises a combination of a HDAC3 inhibitor, for example a selective HDAC3 inhibitor and a MKK1/2 inhibitor. In a related embodiment, an illustrative embodiment comprises a combination of a HDAC3 inhibitor, for example a selective HDAC3 inhibitor and MET mutation inhibitor. In a related embodiment, an illustrative combination comprises an HDAC3 inhibitor, for example, a selective HDAC3 inhibitor, and one or more MAPK inhibitors, wherein the one or more MAPK inhibitors is selected from dabrafenib, encorafenib, trametinib, tovorafenib, vemurafenib, PLX4720, GSK2118436 (GSK436), cobimetinib, RAF265 (also called CHIR-265, sorafenib, Tipifarnib, U0126 (EMD Biosciences (662005)), PD184352 (Axon Medchem (1368)), and SB203580 (EMD Biosciences (55389)). In these exemplified combination treatments, the HDAC3 inhibitor is selected from Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, UF010, Tucidinostat (Chidamide), Citarinostat (ACY-241), Domatinostat (4SC-202), and HPOB and the one or more MAPK inhibitors is selected from: dabrafenib, encorafenib, trametinib, tovorafenib, vemurafenib, PLX4720, GSK2118436 (GSK436), cobimetinib, RAF265 (also called CHIR-265, sorafenib, Tipifarnib, U0126 (EMD Biosciences (662005)), PD184352 (Axon Medchem (1368)), and SB203580 (EMD Biosciences (55389)). In other embodiments, the combination comprises a HDAC3 inhibitor, and at least one MAPK inhibitor selected from: dabrafenib, encorafenib, trametinib, tovorafenib, vemurafenib, PLX4720, GSK2118436 (GSK436), cobimetinib, RAF265 (also called CHIR-265, sorafenib, Tipifarnib, U0126 (EMD Biosciences (662005)), PD184352 (Axon Medchem (1368)), and SB203580 (EMD Biosciences (55389)). In another embodiment, the combination comprises the selective HDAC3 inhibitor RGFP966, and the MAPK inhibitor is selected from: dabrafenib, encorafenib, trametinib, tovorafenib, vemurafenib, PLX4720, GSK2118436 (GSK436), cobimetinib, RAF265 (also called CHIR-265, sorafenib, Tipifarnib, U0126 (EMD Biosciences (662005)), PD184352 (Axon Medchem (1368)), and SB203580 (EMD Biosciences (55389)). In another example, the combination comprises the selective HDAC3 inhibitor RGFP966, and the one or more MAPK inhibitors is selected from dabrafenib, trametinib, vemurafenib, and PLX4720. In another example, the combination comprises the selective HDAC3 inhibitor RGFP966, and a MAPK inhibitor is selected from: dabrafenib, trametinib, and vemurafenib. In another example, the combination comprises the selective HDAC3 inhibitor RGFP966, and the MAPK inhibitor is dabrafenib. In another example, the combination comprises the selective HDAC3 inhibitor RGFP966, and the MAPK inhibitor is trametinib. In another example, the combination comprises the selective HDAC3 inhibitor RGFP966, and the MAPK inhibitor is vemurafenib. In another example, the combination comprises the selective HDAC3 inhibitor RGFP966, and the MAPK inhibitor is PLX4720. In another example, the combination comprises the selective HDAC3 inhibitor RGFP966, and the one or more MAPK inhibitors are dabrafenib and trametinib.

In other exemplified combination treatments, the HDAC3 inhibitor is selected from Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, UF010, Tucidinostat (Chidamide), Citarinostat (ACY-241), Domatinostat (4SC-202), and HPOB and the secondary active agent is a chemotherapeutic agent. In another exemplified example, the In these exemplified combination treatments, the HDAC3 inhibitor is selected from Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, UF010, Tucidinostat (Chidamide), Citarinostat (ACY-241), Domatinostat (4SC-202), and HPOB and the one or more chemotherapeutic agents are selected from: cisplatin, carboplatin, 5-fluorouracil, cyclophosphamide, oncovin, vincristine, prednisone, or rituximab, mechlorethamine, cyclophosphamide, ifosfamide, melphalan, chlorambucil, carmustine, lomustine, semustine, thriethylenemelamine, triethylene thiophosphoramide, hexamethylmelamine altretamine, busulfan, triazines dacarbazine, methotrexate, trimetrexate, fluorodeoxyuridine, gemcitabine, cytosine arabinoside, 5-azacytidine, 2,2′-difluorodeoxycytidine, 6-mercaptopurine, 6-thioguanine, azathioprine, 2′-deoxycoformycin, erythrohydroxynonyladenine, fludarabine phosphate, 2-chlorodeoxyadenosine, camptothecin, topotecan, irinotecan, paclitaxel, vinblastine, vincristine, vinorelbine, docetaxel, estramustine, estramustine phosphate, etoposide, teniposide, mitoxantrone, mitotane, aminoglutethimide or combinations thereof. In further examples the HDAC3 inhibitor is a selective HDAC3 inhibitor, and the one or more chemotherapeutic agents are selected from: cisplatin, carboplatin, 5-fluorouracil, cyclophosphamide, oncovin, vincristine, prednisone, or rituximab, mechlorethamine, cyclophosphamide, ifosfamide, melphalan, chlorambucil, carmustine, lomustine, semustine, thriethylenemelamine, triethylene thiophosphoramide, hexamethylmelamine altretamine, busulfan, triazines dacarbazine, methotrexate, trimetrexate, fluorodeoxyuridine, gemcitabine, cytosine arabinoside, 5-azacytidine, 2,2′-difluorodeoxycytidine, 6-mercaptopurine, 6-thioguanine, azathioprine, 2′-deoxycoformycin, erythrohydroxynonyladenine, fludarabine phosphate, 2-chlorodeoxyadenosine, camptothecin, topotecan, irinotecan, paclitaxel, vinblastine, vincristine, vinorelbine, docetaxel, estramustine, estramustine phosphate, etoposide, teniposide, mitoxantrone, mitotane, aminoglutethimide or combinations thereof. In another illustrative embodiment, the combination comprises a therapeutically effective dose of a selective HDAC3 inhibitor, for example, RGFP966, and the one or more chemotherapeutic agents are selected from: cisplatin, carboplatin, 5-fluorouracil, cyclophosphamide, oncovin, vincristine, prednisone, or rituximab, mechlorethamine, ifosfamide, melphalan, chlorambucil, carmustine, lomustine, semustine, thriethylenemelamine, triethylene thiophosphoramide, hexamethylmelamine altretamine, busulfan, triazines dacarbazine, methotrexate, trimetrexate, fluorodeoxyuridine, gemcitabine, cytosine arabinoside, 5-azacytidine, 2,2′-difluorodeoxycytidine, 6-mercaptopurine, 6-thioguanine, azathioprine, 2′-deoxycoformycin, erythrohydroxynonyladenine, fludarabine phosphate, 2-chlorodeoxyadenosine, camptothecin, topotecan, irinotecan, paclitaxel, vinblastine, vincristine, vinorelbine, docetaxel, estramustine, estramustine phosphate, etoposide, teniposide, mitoxantrone, mitotane, aminoglutethimide or combinations thereof. In some embodiments, the combination comprises therapeutically effective doses of RGFP966, and the one or more chemotherapeutic agents selected from: vincristine, cyclophosphamide, and methotrexate.

In each of the foregoing combinations, the dose of the HDAC3 inhibitor is a therapeutically or effective dose, and the dose of the secondary agent is a therapeutically or effective dose. In some embodiments, the HDAC3 inhibitor is dosed and administered to a subject with LCH or LCS or is likely to develop LCH or LCS in accordance with a prior determined therapeutic regimen as determined in a clinical trial for the treatment of cancer, neurological disease or cardiovascular disease. Similarly, the dose of the secondary active agent, for example, a chemotherapeutic agent, an immunomodulating agent, a MAPK inhibitor or combinations thereof, is a therapeutically or effective dose, as previously deemed therapeutically active and safe in accordance with a prior determined therapeutic regimen as determined in a clinical trial for the treatment of cancer.

### Definitions

At various places in the present specification, features or functions of the compositions of the present disclosure are disclosed in groups or in ranges. It is specifically intended that the present disclosure include each and every individual sub combination of the members of such groups and ranges.

As used herein, the term “approximately” or “about,” as applied to one or more values of interest, refers to a value that is similar to a stated reference value. In certain embodiments, the term “approximately” or “about” refers to a range of values that fall within 25, 20, 19, 18, 17, 16, 15, 14, 13, 12, 11, 10, 9, 8, 7, 6, 5, 4, 3, 2, 1, or less in either direction (greater than or less than) of the stated reference value unless otherwise stated or otherwise evident from the context (except where such number would exceed 100 of a possible value).

As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

The term “cancer” as used herein refers a broad group of various diseases characterized by the uncontrolled proliferation of abnormal cells in the body. Unregulated cell division and growth results in the formation of malignant tumors that invade neighboring tissues, which can ultimately metastasize to distant parts of the body through the lymphatic system or bloodstream.

The term “co-stimulating molecule” as used herein refers to a group of immune cell surface receptor/ligands and soluble molecules, which engage T cells and APCs. T cell stimulatory signals enhance the T cell response to receptor (TCR) recognition of antigen/MIC complex (pMHC) on APCs, and APC stimulatory molecule may induce differentiation or the expression or secretion of immunity priming molecules.

The term “cytokines”, as used herein, refers to a family of small soluble factors with pleiotropic functions that are produced by many cell types that can influence and regulate the function of the immune system.

The term “delivery” as used herein refers to the act or manner of delivering a compound, substance, entity, moiety, cargo or payload. A “delivery agent” refers to any agent which facilitates, at least in part, the in vivo delivery of one or more substances (including, but not limited to a compound and/or composition of the present disclosure) to a cell, subject or other biological system cells.

As used herein, the term “feature” refers to a characteristic, a property, or a distinctive element.

As used herein, the term “formulation” includes at least a compound and/or composition of the present disclosure and a delivery agent.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

The term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments, a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N, N′-dimethylformamide (DMF), N, N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

As used herein, the terms “treatment” or “treating” denote an approach for obtaining a beneficial or desired result including and preferably a beneficial or desired clinical result. Such beneficial or desired clinical results include, but are not limited to, one or more of the following: reducing the proliferation of (or destroying) LCH and LCS cells, shrinking the size of the lresions containing the LCH and LCS or precursors thereof, decreasing symptoms resulting from the disease, increasing the quality of life of those suffering from the disease, decreasing the dose of other medications required to treat the disease, delaying the progression of the disease, and/or prolonging survival of individuals.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., therapeutic or active ingredient; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure. The present disclosure is further illustrated by the following nonlimiting examples.

### EXAMPLES

### Example 1. HDAC3 is Required for Embryonic Development of LCs

The role of HDAC3 in LCs precursor differentiation was examined during embryonic development by generating Csf1riCreHdac3fl/fl conditional knockout (cKO) mice, in which HDAC3 is deficient in Csf1r-expressing myeloid cells, including TRMs and monocytes.

### Methods

HDAC3 mRNA expression was measured in WT and HDAC3 conditional knock-out (Csf1riCreHdac3fl/fl) embryonic and adult mice using quantitative Real-time PCR (qRT-PCR)

The Csf1riCre mouse strain (#021024) was purchased from Jackson Laboratory (Bar Harbor, ME). Hdac3fl/fl mice were provided by Scott W. Hiebert. All transgenic mice were completely viable with a normal Mendelian frequency and backcrossed with C57BL/6 mice for 6 generations for this study. Male and female mice from 6-18 weeks of age were used. All experiments included age- and sex-matched littermate controls. Embryonic stage 0.5 days was estimated from the day of vaginal plug observance.

### Collection of Cell Samples

Mouse embryos were obtained from plug-staged pregnant females sacrificed by CO2 exposure. Embryos ranging from embryonic days E10.5 to E18.5 were removed from the uterus and washed in cold PBS followed by decapitation. The skin samples from embryos at E18.5 and newborn (P0) were treated with dispase II the same as described above for the adult skin preparation. Skin from E10.5 to E16.5 embryos was collected, minced, and incubated at 37° C. for 30 min in PBS containing 3% FBS, 1 mg/ml collagenase D (Roche, Basel, Switzerland), and 0.01% DNase I. The erythrocytes were lysed as described above. All cell suspensions were passed through a 70 μm cell strainer (BD Biosciences, San Jose, CA).

### Genotyping

Hdac3fl/fl mice were genotyped by conventional protocols using the following PCR primer pair: forward, SEQ ID NO: 23 (5′-GGA CAC AGT CAT GAC CCG GTC-3′); reverse, SEQ ID NO: 24 (5′-CTC TGG CTT CTG CTA TGT CAA TG-3′). The floxed allele produced a 504-bp PCR product whereas the wild-type (WT) allele resulted in a 464-bp PCR product.

### RNA Isolation and Quantitative RT-PCR

RNA was isolated from the sorted cells with GenElute Total RNA Purification Kit (Sigma Aldrich) and converted to cDNA with High Capacity cDNA Reverse Transcription Kits (Applied Biosystems, Foster City, CA). For quantitative PCR, reactions were performed using FastStart Universal SYBR Green Master (ROX, Roche) and carried out using QuantStudio 7 Flex Real-Time PCR System (Applied Biosystems). Data were collected using QuantStudio 7 Flex Real-Time PCR System software version 1.2 (Applied Biosystems) and analyzed using Microsoft Excel 2016 (Microsoft, Redmond, Washington). The full list of primers used in the Examples are shown in Table 1 below. Samples were normalized to β-actin or GAPDH expression.

TABLE 1
Primers used for qRT-PCR

| Gene | SEQ | | SEQ | |
|---|---|---|---|---|
| (Accession) | ID | Forward (5′-3′) | ID | Reverse (5′-3′) |
| Actb | 1 | CAACGAGCGGTTCCGATG | 2 | GCCACAGGATTCCATACCCA |
| Ahr | 3 | CGCTGAAACATGAGCAAATTGG | 4 | ACAGCTTAGGTGCTGAGTCACAGG |
| Anapc2 | 5 | CGCCTCTATGGTCGTTTCTT | 6 | GCTATCTAGCTCACCCTCTAGT |
| Ccna2 | 7 | CACTGACACCTCTTGACTATCC | 8 | CGTTCACTGGCTTGTCTTCTA |
| Ccnb2 | 9 | CTCTGCAAGATCGAGGACATAG | 10 | TGCCTGAGGTACTGGTAGAT |
| Cdc25b | 11 | CACCTCTCGGTCTTTGAGTTT | 12 | TGTGCATGGTCTGTGTAAGAG |
| Cdca3 | 13 | AGCGAAGTATTGGAGACAGAAG | 14 | GAAGACAGGTCCAGGTCATAAA |
| Chek2 | 15 | CCTGAAAGAAGCCACCTGTAA | 16 | GTCCCGATGTATGATCCCATTT |
| Csf1r | 17 | ATCCCCCTAAACAGTGACTTCC | 18 | GGTCAGATTATTCCAGCC |
| Foxm1 | 19 | CCAGGCTGGAAGAACTCTATTC | 20 | TTGGGTGAATGGTCCAGAAG |
| Gapdh | 21 | GGTGAAGGTCGGTGTGAACG | 22 | TGTAGACCATGTAGTTGAGGTCA |
| Hdac3 | 23 | AGCTGGACACCCAATGAAAC | 24 | TGTTGCTCCTTGCAGAGATG |
| Id2 | 25 | AAAACAGCCTGTCGGACCAC | 26 | CTGGGCACCAGTTCCTTGAG |
| Kifc1 | 27 | TGCCTTTGCACCCTTTCT | 28 | CCTTCAGTTCTACGTTCCTCTTC |
| Kat2b | 29 | AGGAGACCTCCAGCAGATAAT | 30 | CCATCTCTTCCTCTGACACATTC |
| Lamtor2 | 31 | TCTGGGCCGCGTATGATAGG | 32 | CACGCTGTTATGATGCTGCTACTT |
| huLangerinCre | 33 | GAGGCAAATGATTGGCATTCT | 34 | CTGGAAAATTCAAGAAGAGCC |
| Langerin-WT | 35 | GTTGCAGCAAATTCCAACCC | 36 | GGCCAGGAGAAACTACAAGTC A |
| Mad2l1 | 37 | GGACTCACCTTGCTTACAACTA | 38 | GATGACCACCACCAGTTTCT |
| Nostrin | 39 | GATGCCAAGAGCCAGAAAGA | 40 | CACTGACGACAGTTTGTAGGAG |
| Plau | 41 | CGCACACTGCTTCATTCAAC | 42 | GCTCCACCTCAAACTTCATCT |
| Rgcc | 43 | AGCTTCACCTTCAGTGATGAG | 44 | GTGTCACCTAATTTGGCTTTCC |
| Runx3 | 45 | GGTTCAACGACCTTCGCTTC | 46 | CACGGTCACCTTGATGGCT |
| Spi1 | 47 | CATAGCGATCACTACTGGGATTTC | 48 | GGTTCTCAGGGAAGTTCTCAAA |
| Stk11 | 49 | GCCAACGTCAAGAAGGAGAT | 50 | CGCAGTACTCCATCACCATATAC |
| Trerf1 | 51 | GACAAGTGGACATCCCTAGAAAG | 52 | TGTCTTGGACTTCACCATCTTC |
| Zeb1 | 53 | CTTACGGATTCACAGTGGAGAG | 54 | GTGAGCTATAGGAGCCAGAATG |

LC precursor biomarkers were measured in fetal skin by immunolabeling and flow cytometry analysis. The frequency of LC precursors in fetal skin were identified by immunolabeling for F4/80+CD11b+ cell markers and sorting by flow cytometry. The frequency of macrophages in fetal skin were identified by immunolabeling for F4/80hiCD11lo cell markers and sorting by flow cytometry. The frequency of monocytes in fetal skin were identified by immunolabeling for F4/80loCD11bhi cell markers and sorting by flow cytometry.

### Flow Cytometry Cell Sorting

Biomarkers of Langerhan cells precursors (CD11b and F4/80) in fetal skin were immunolabeled and sorted by flow cytometry. Single-cell suspensions were centrifuged at 450×g for 7 min, resuspended in 4° C. staining buffer (1×PBS containing 2% FBS), and placed in 96-well round-bottom plates. After incubation with purified anti-FcγRII/III antibody (clone 2.4G2) at 4° C. for 15 min, cells were stained at 4° C. for 30 min with a mixture of fluorescent surface antibodies. For intracellular staining, fresh cells with surface staining were fixed with Foxp3/permeabilization buffer (Concentrate: Diluent solution=1:3, eBioscience/Thermo Fisher Scientific) at 4° C. for 25 min and stained with intracellular antibodies in 1× permeabilization buffer (eBioscience/Thermo Fisher Scientific) at 4° C. for 30 min. The full list of antibodies used are shown in Table 2. The stained samples were then acquired on a FACSAria™ II or FACSCelesta™ flow cytometer (BD Biosciences) and data were analyzed using FlowJo 10.5.3 (BD Biosciences). The epidermal CD45+MHC-II+ LCs from adult mice and CD45+F4/80+CD11b+epidermal LC precursors from embryos at E14.5, E16.5, and E18.5 were sorted on a FACSAria™ II flow-cytometer. The purity of the sorted populations was >95%. Spleen CD4+ T cells from OT-II mice were enriched using Dynal® mouse CD4-negative selection kit (Thermo Fisher Scientific) with >85% purity.

TABLE 2
Antibodies used for flow cytometry and immunofluorescence

| Antibody | Clone | Conjugate | Company | Catalog# | Concentration |
|---|---|---|---|---|---|
| CD11b | M1/70 | PerCP/Cy5.5 | eBioscience | 45-0112-82 | 0.05 μg/100 μL |
| | | eBioscience | | | |
| CD11b | M1/70 | APC-CY7 | Biolegend | 101226 | 0.1 μg/100 μL |
| CD11b | M1/70 | FTIC | eBioscience | 11-0112-85 | 0.25 μg/100 μL |
| CD207 (langerin) | 4C7 | PE | Biolegend | 144204 | 0.1 μg/100 μL |
| CD207 (langerin) | 4C7 | APC | Biolegend | 144206 | 0.2 μg/100 μL |
| CD207 (langerin) | 929F3.01 | Alexa Fluor 488 | Dendritics | DDX0362 | 1.0 μg/100 μL |
| CD4 | GK1.5 | FITC | Tonbo | 35-0041-U500 | 0.25 μg/100 μL |
| CD8 | 53-6.7 | PE | eBioscience | 12-0081-83 | 0.1 μg/100 μL |
| CD45 | 30-F11 | APC | Tonbo | 20-0451-U100 | 0.01 μg/100 μL |
| CD45 | 30-F11 | FITC | Tonbo | 35-0451-U500 | 0.5 μg/100 μL |
| CD45 | 30-F11 | PE | Tonbo | 50-0451-U100 | 0.1 μg/100 μL |
| CD45 | 30-F11 | APC-Cy7 | BioLegend | 103116 | 0.05 μg/100 μL |
| CD45 | 30-F11 | BV785 | BioLegend | 103149 | 0.005 μg/100 μL |
| CD45.1 | A20 | APC | Tonbo | 20-0453-U100 | 0.1 μg/100 μL |
| CD45.1 | A20 | FITC | Tonbo | 35-0453-U500 | 0.1 μg/100 μL |
| CD45.1 | A20 | APC-ef780 | eBioscience | 47-0453-82 | 0.2 μg/100 μL |
| CD45.2 | 104 | PE | eBioscience | 12-0454-82 | 0.2 μg/100 μL |
| CD45.2 | 104 | PerCP/Cy5.5 | eBioscience | 45-0454-82 | 0.1 μg/100 μL |
| | | eBioscience | | | |
| CD45.2 | 104 | FITC | Tonbo | 35-0454-U500 | 0.25 μg/100 μL |
| CD80 | 16-10A1 | FITC | Tonbo | 35-0801-U500 | 0.25 μg/100 μL |
| CD86 | GL-1 (GL1) | PerCP/Cy5.5 | BioLegend | 105026 | 0.1 μg/100 μL |
| | | BioLegend | | | |
| F4/80 | BM8 | eFluor 450 | eBioscience | 48-4801-82 | 0.05 μg/100 μL |
| F4/80 | BM8.1 | APC | Tonbo | 20-4801-U100 | 0.1 μg/100 μL |
| HDAC3 | Y415 | Purified | Abcam | ab32369 | 0.075 μg/100 μL |
| I-A/E | M5/114.15.2 | PE | eBioscience | 12-5321-83 | 0.02 μg/100 μL |
| I-A/E | M5/114.15.2 | APC | eBioscience | 17-5321-82 | 0.1 μg/100 μL |
| I-A/E | M5/114.15.2 | FITC | eBioscience | 11-5321-82 | 0.05 μg/100 μL |
| I-A/E | M5/114.15.2 | eFluor 450 | eBioscience | 48-5321-82 | 0.04 μg/100 μL |
| IFN-γ | XMG1.2 | eFluor 450 | Invitrogen | 48-7311-82 | 0.1 μg/100 μL |
| IL-17A | TC11-18H10.1 | Alexa Fluor 700 | BioLegend | 506914 | 0.25 μg/100 μL |
| IL-17A | eBio17B7 | APC | eBioscience | 17-7177-81 | 0.05 μg/100 μL |
| Ki-67 | SolA15 | PE | eBioscience | 12-5698-82 | 0.06 μg/100 μL |
| Ki-67 | SolA15 | PE-Cy7 | eBioscience | 25-5698-82 | 0.06 μg/100 μL |
| Ki-67 | 16A18 | Alexa Fluor 700 | Biolegend | 652420 | 0.125 μg/100 μL |
| TCRb | H57-597 | PerCP/Cy5.5 | BioLegend | 109228 | 0.2 μg/100 μL |
| | | BioLegend | | | |
| TNF | mp6-xt22 | APC-Cy7 | Biolegend | 506344 | 0.1 μg/100 μL |
| Annexin V | | eFluor 450 | Invitrogen | 48-8006-69 | 3.0 μL/100 μL |
| Goat anti-rabbit IgG | | Alexa Fluor 488 | Life | A11034 | 0.1 μg/100 μL |
| | | | Technologies | | |

### Results

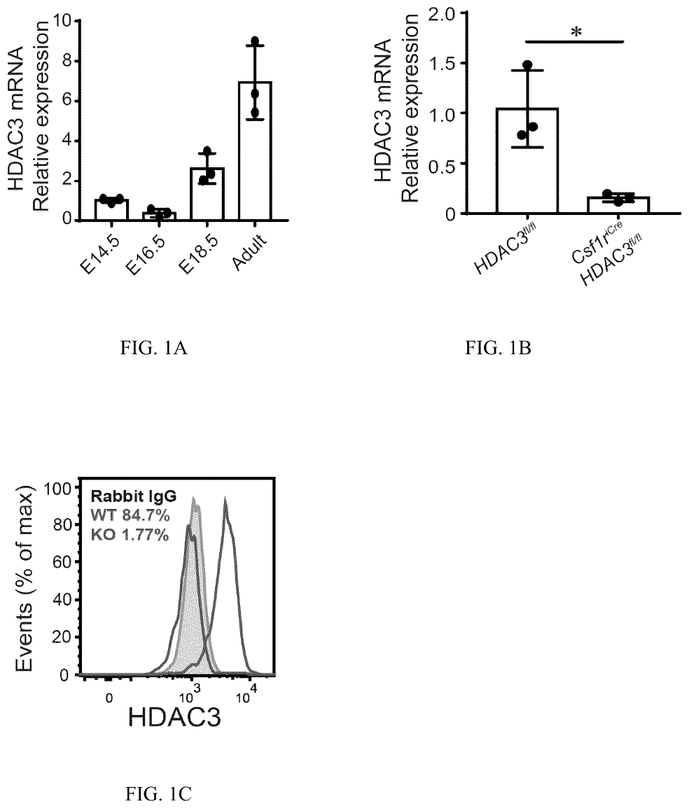

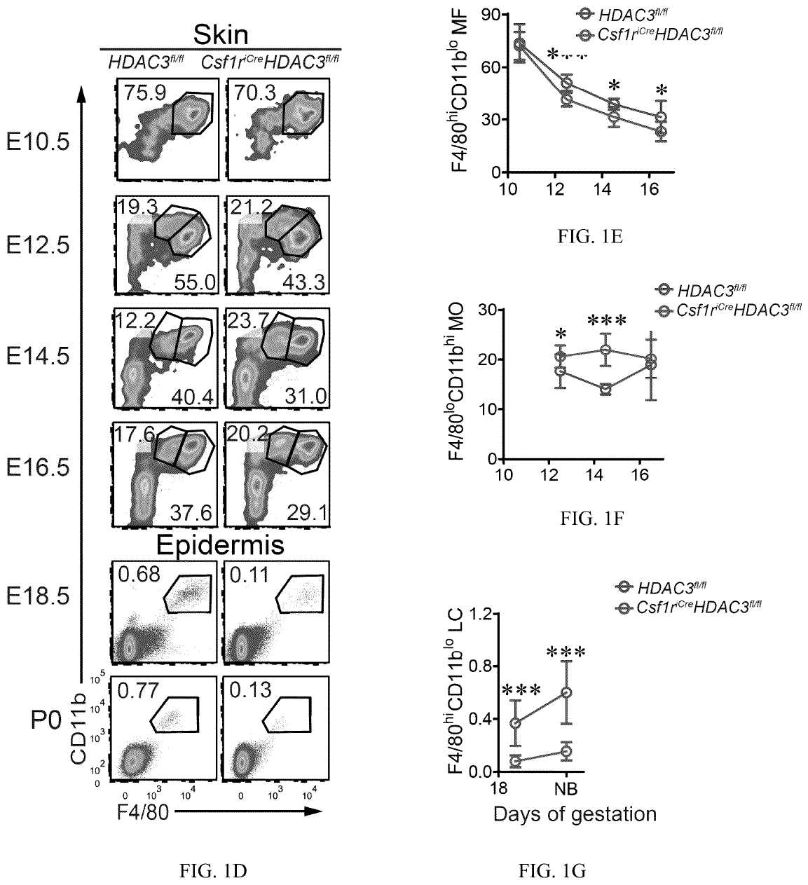

The HDAC3 knock-out mouse is completely viable. HDAC3 mRNA expression increases with developmental age and is highest in LC precursors in the adult mouse (FIG. 1A). To investigate the role of HDAC3 in LC ontogeny and homeostasis, the expression pattern of HDAC3 in skin TRMs and epidermal LC precursors was examined during embryonic development and in immature adult epidermal LCs. As shown in FIG. 1A, HDAC3 mRNA was detectable in skin TRMs at E14.5 and E16.5. Its expression was moderately increased (˜3-fold) in epidermal LC precursors at E18.5 and was dramatically augmented (˜7-fold) in immature LCs from adult mice. These results suggest that HDAC3 is involved in LC embryonic development and maintenance after birth.

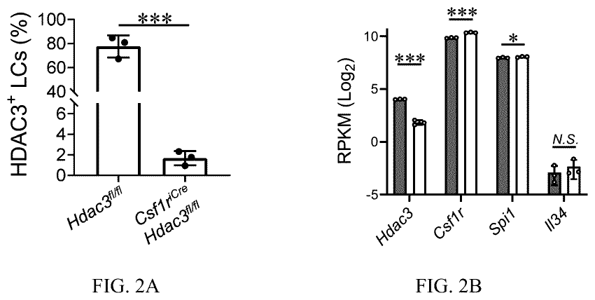

The expression levels of HDAC3 mRNA and protein were robustly reduced (>80%) in LC precursors from the HDAC3 cKO newborns measured by qRT-PCR (FIG. 1B) and flow cytometry (FIG. 1C, FIG. 2A), respectively. TRMs and fetal monocytes were examined in the skin from Csf1riCreHdac3fl/fl cKO mouse embryos and Hdac3fl/fl WT littermates between E10.5 and E16.5. Since epidermal sheets can be separated from the dermal layer starting from E17.5, LC precursor frequency was examined in the epidermis at E18.5 and at P0, immediately after birth. There was no significant difference in the frequencies of F4/80hiCD11blo TRMs and F4/80loCD11bhi fetal monocytes in the skin of cKO and WT embryos at E10.5 (FIGS. 1D, 1E, and 1F). However, the frequency of skin TRMs was modestly but significantly reduced in HDAC3 cKO embryos between E12.5 and E16.5 compared to WT. At E18.5 and P0, the epidermal LC precursors were almost completely abrogated in HDAC3 cKO embryos compared to WT (FIG. 1G). In contrast, skin fetal monocytes showed a relative increase at E12.5 and E14.5 or remained unaltered at E16.5 in the absence of HDAC3. Collectively, these results suggest that HDAC3 is critical for the development of skin TRMs and LC precursors during embryogenesis.

### Example 2. HDAC3 Protein and Gene Expression is Significantly Reduced in LC Precursor Cells from HDAC3-Deficient Mice

Example 2 examined the level of HDAC3 protein was determined in Hdac3fl/fl and Csf1riCreHdac3fl/fl newborns (P0) by flow cytometry. The level HDAC3 gene and genes related to LC development were determined by sequencing bulk RNA derived from F4/80+CD11b+LC precursors sorted from the fetal epidermis of Hdac3fl/fl and Csf1riCreHdac3fl/fl newborn (P0) pups.

### Methods

Newborn pups were obtain from the Hdac3fl/fl and Csf1riCreHdac3fl/fl mice described in Example 1. The method of isolating epidermal cells and their detection by immunolabeling and flow cytometry was performed as described in Example 1. HDAC3 and genes related to LC precursor development (Csf1r, Spi1, and 1134) were quantified by Bulk RNA sequencing.

### Bulk RNA Sequencing

cDNA synthesis and preamplification of total RNA from the sorted cells were performed using the SMART-Seq v4 Ultra Low Input RNA Kit (Clontech, Mountain View, CA) following a standard protocol as described previously. Briefly, cDNA was pre-amplified, sheered and repaired, and the end-repaired DNA fragments were ligated to adaptors. After DNA cleanup, final library amplification was performed followed by gel purification. Sequencing was performed by the DNA sequencing core at University of Michigan using a 50 bp single end read setup on the Illumina Hiseq 4000 platform.

### Results

As expected, HDAC3 protein and gene expression was significantly decreased in Csf1riCreHdac3fl/fl mice compared to Hdac3fl/flmice (FIGS. 2A, 2B). The expression of genes Csf1r and Spi1 are slightly reduced, while the expression of 1134 is unchanged, in Csf1riCreHdac3fl/fl mice compared to Hdac3fl/flmice (FIG. 2B). The results indicate that the loss of HDAC3 expression does not appreciably affect the expression of three genes related to LC precursor development.

### Example 3. HDAC3 Controls Embryonic LC Development Through Regulation of Cell Proliferation

Example 3 examined the differentially expressed gene profile by bulk RNA-seq analysis of LC precursors from the fetal epidermis of Csf1riCreHdac3fl/fl and WT to understand the developmental effects caused by HDAC3 deficiency. The direct downstream targets of HDAC3 were also identified by ChIP-seq analysis from WT mice during LC development.

### Methods

The mice were obtained as described in Example 1. Fetal epidermis was obtained from E18.5 Hdac3fl/fl and Csf1riCreHdac3fl/fl mice as described in Example 1. LC precursors expressing F4/80+CD11b+ were sorted by flow cytometry as described in Example 1, and the Bulk RNA sequencing was performed as described in Example 2.

ChIP sequencing was performed by first cross-linking HDAC3 to genomic DNA, then shearing the DNA, and immunoprecipitating the HDAC3 bound DNA. The immunoprecipated DNA is then unlinked and amplified for next generation sequencing.

### ChIP Sequencing

Cell pellets from the sorted LCs (0.72×10⁶ and 1.2×10⁶ cells from pooled mice) from WT adult mice were directly frozen at −80° C. Two biological independent samples were then submitted to EpiGentek (Farmingdale, NY) for ChIP, library preparation, and sequencing. Briefly, the cells were fixed with 1% formaldehyde and chromatin was isolated using a CHROMAFLASH™ Chromatin Extraction Kit. Chromatin was sheared to 100-300 bp fragments using the Episonic2000 Sonicating System. ChIP was performed on the chromatin using an antibody against HDAC3 (Abcam, #ab7030, polyclonal, 1 μg/ChIP), and input DNA (without immunoprecipitation) was used to determine background. After adapter linking, the libraries were amplified using indexed primers followed by purification. Ten nanomolar of each sample library was provided for next generation sequencing on a HiSeq 2500.

### ChIP-qPCR

The ChIP assay was performed on 4×10⁶ cells from 80-90% confluent XS-106 cell cultures for each immunoprecipitation using an SimpleChIP Plus Enzymatic Chromatin IP kit (Cell Signaling, Danvers, MA, #9005) and 10 μl of anti-Histone H3 antibody (Cell Signaling, #4620, clone D2B12, 10 μl/500 μl 1×ChIP buffer), 2 μg of anti-HDAC3 antibody (Abcam, #ab32369, clone Y415, 2 μg/ml), and 1 μg of rabbit IgG (Abcam, #ab171870, polyclonal, 1 μg/ml) per reaction. DNA-relative enrichment was determined by normalization to the rabbit IgG control background signals. All ChIP experiments were obtained from independent chromatin preparations, and all quantitative real-time PCR reactions were performed in duplicates for each sample on each amplicon. Mouse RPL30 gene (primers from Cell Signaling, #7015) was used as a positive control for DNA quantification. Primers for the ChIP-qPCR are listed in Table 3.

TABLE 3
Primers used for ChIP-qPCR

| Gene | SEQ | | SEQ | |
|---|---|---|---|---|
| (Accession) | ID | Forward (5′-3′) | ID | Reverse (5′-3′) |
| Cdk14-ChIP | 55 | GAGATCATGGTGTTAAGCAACAAA | 56 | ACACACACACACACACACA |
| Cyrib-ChIP | 57 | GGAGAAGTGAAAGGAGGGTTT | 58 | TCTCTCTCTCTCTCTCTCTCTCT |
| Kif16b-ChIP | 59 | AACCTCCCTTCTGATAAATAGTTGA | 60 | GTGCTACCAAAGAAGATGAATGG |
| Mad2l1-ChIP | 61 | GCCTACATGCTATCTCCATTGT | 62 | CTCTCTCTCTCTGTCTGTCTTCT |
| Nostrin-ChIP | 63 | CTCTCTAAATACCAGTCTTCCTTCTT | 64 | GGAGGAAAGAAAGGGAAAGGA |
| Plau-ChIP | 65 | CAGTCTGCCCATGCAATTATC | 66 | CAGAAAGAGTGTGTGTATGTGTG |
| Trerf1-ChIP | 67 | TAACTGGCATTCTGGGTAATGG | 68 | CACCTCAGGATTTGTGGTAGTG |
| Zeb1-ChIP | 69 | CTAAGGTTTCTCCTACTGAGAAAGAA | 70 | GCCTTGCTTACAATGGAATGG |
| Id2 intron + | 71 | GAAGCCACAGTTTGAGCATTTA | 72 | GAGACTGGGTTTGGGAAGTT |
| 1.3 kb | | | | |
| Runx3 intron + | 73 | TGAAGCCCAGAGACTCAGAT | 74 | TACCAGAGGACCCACATTCT |
| 12 kb | | | | |
| Runx3 intron + | 75 | CACAGCTCAAGCAGGGTATAG | 76 | GGAGACCACAGAGGACTTGTA |
| 35 kb | | | | |

### Bulk RNA-Seq and ChIP-Seq Data Processing

The bulk RNA-Seq data and ChIP-Seq data was integrated to identify genes present in both datasets. The RNA-seq reads were aligned to the mouse GRCm38/mm10 reference genome and expression levels for each sample were quantified and normalized using Biomedical Genomics Workbench 5.0 (QIAGEN, Germantown, MD). Gene ontology (GO) biological process enrichment analysis was performed using DAVID Bioinformatics Resources 6.8. Gene Set Enrichment Analysis (GSEA) was performed using GSEA software 4.0.3. Further pathway analysis was performed using the Ingenuity Pathway Analysis (IPA) (QIAGEN Bioinformatics, version 42012434). Raw ChIP-seq reads were quality checked using the software FastQC version v0.10.1. The quality checked reads were then mapped to the mouse mm9 genome sequence using Bowtie (v1.0.0) and only uniquely mapped reads were retained. Mapping results of each ChIP and the input sample were subjected to ChIP-enriched peak calling using the MACS (v2.1.0). Peak annotation was conducted using ChIPseeker (v1.24.0). The peaks identified (FDR<0.05) simultaneously from LC duplicates were used for further analysis. Venn diagram was created through an online tool (http://bioinformatics.psb.ugent.be/webtools/Venn/).

### Statistical Analysis

No statistical method was used to predetermine sample size. All results are displayed as mean±SD or mean±SEM, and two-tailed Student's t-test was used to calculate the P values using GraphPad Prism 8.4.3 software (GraphPad, La Jolla, CA) unless otherwise specified. Statistical significance is displayed as below: N.S., not significant; *P<0.05; **P<0.01; ***P<0.001.

Bulk RNA-seq (three biologically independent samples/group) and ChIP-seq (two biologically independent samples/group) were performed one time. Immunofluorescence, bone marrow chimera, qRT-PCR experiments were performed independently twice with similar results. All other experiments were performed independently at least three times with similar results.

### Results

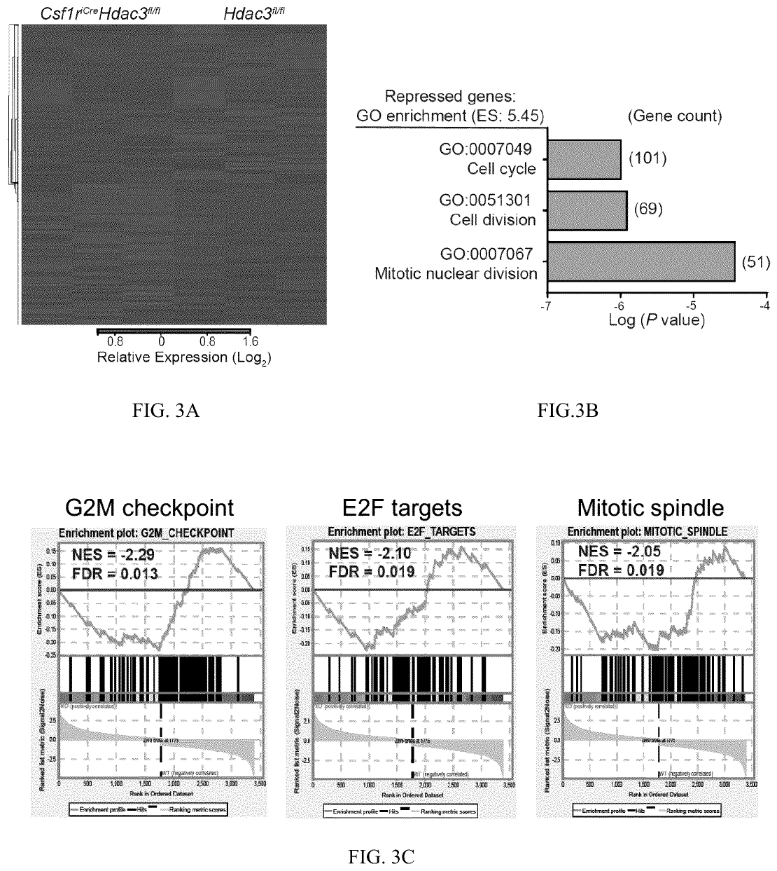

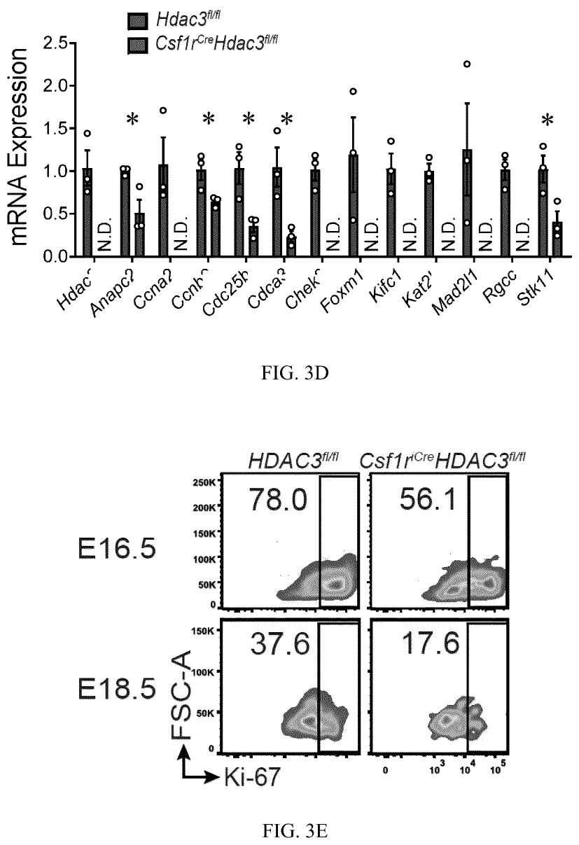

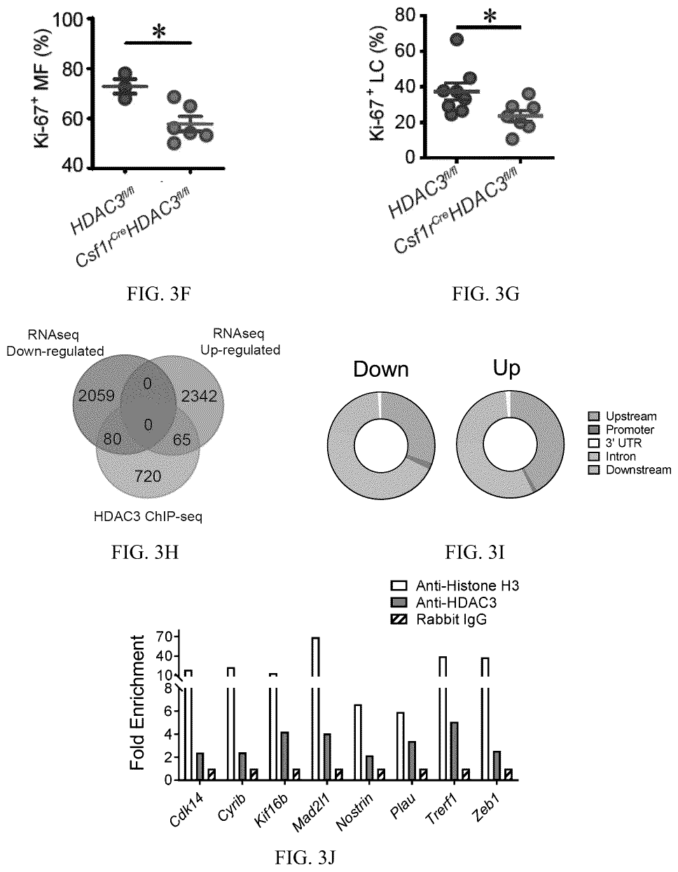

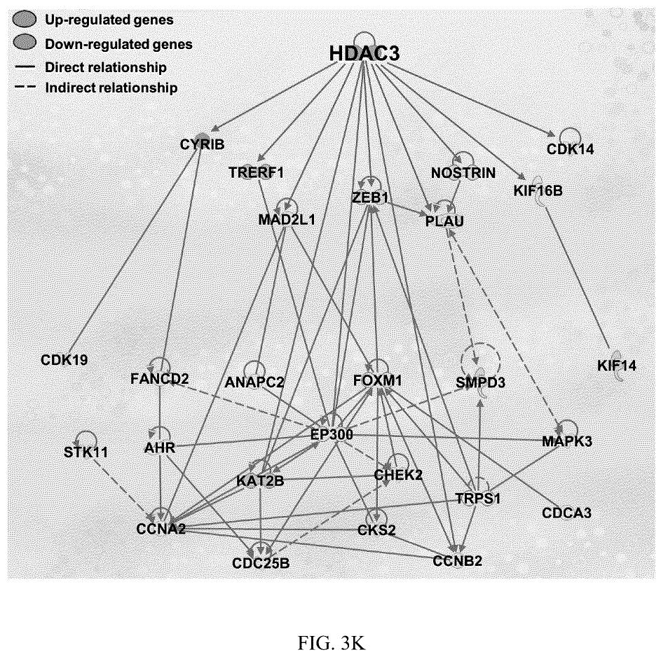

A total of 4,546 differentially expressed genes (FDR<0.05) were identified in the LC precursors from HDAC3 cKO mice compared to WT, including 2,139 down-regulated and 2,407 up-regulated genes, are shown in the heat map (FIG. 3A). Although Csf1r, Spi1 and 1134 are essential for development of skin TRMs and LCs, none of them were impaired in LC precursors from HDAC3 cKO mice compared to WT (FIG. 2B), suggesting that they were not involved in the developmental defects of LCs caused by HDAC3 deficiency. Gene Ontology (GO) analysis identified several dysregulated biological pathways in the HDAC3 cKO LC precursors, including those for cell cycle, cell division and mitotic nuclear division (FIG. 3B). Additionally, gene set enrichment analysis (GSEA) identified a few gene sets involved in cell proliferation that were impaired in LC precursors lacking HDAC3, including G2M checkpoint, E2F targets, and mitotic spindle (FIG. 3C). The downregulation of cell proliferation-associated genes, such as Ccna2, Ccnb2, Cdc25b, Cdca3, Chek2, Foxm1, Kat2b, and Stk11, in the HDAC3 cKO LC precursors was confirmed by qRT-PCR (FIG. 3D). A flow cytometry analysis showed a significant decrease in the frequencies of Ki-67+ skin macrophages at E16.5 and Ki-67+LC precursors at E18.5 in HDAC3-deficient embryos (FIG. 3E). The frequency of Ki-67+ skin resident macrophages were reduced in embryos from Csf1riCreHDAC3fl/fl compared to HDAC3fl/flmice (FIG. 3F). The frequency of Ki-67+LC precursors were reduced in embryos from Csf1riCreHDAC3fl/fl compared to HDAC3fl/flmice (FIG. 3G). These results suggest that HDAC3 regulates cell proliferation, which could be related to defective embryonic LC development in HDAC3 cKO mice.

The ChIP sequencing analysis identified 865 overlapping genes in 2 ChIP-seq samples. The integration of the bulk RNA-seq data with the ChIP-seq identified a total of 145 genes present in both datasets, including 80 down-regulated genes and 65 up-regulated genes in the HDAC3 cKO LC precursors that could be primary targets of HDAC3 transcriptional regulation (see Venn diagram, FIG. 3H). ChIP-seq analysis showed an enrichment of HDAC3-binding peaks at the enhancer regions located upstream, downstream, or within the introns of the differentially expressed genes (see donut charts, FIG. 3I). ChIP-qPCR using a murine epidermal DC cell line XS-106 cells further confirmed the binding of HDAC3 to the enhancers of genes Cdk14, Cyrib, Kif16b, Mad2l1, Nostrin, Plau, Trerf1, and Zeb1. (FIG. 3J). Ingenuity pathway analysis revealed that these HDAC3 targeted genes either belonged to cell cycle and division pathways or interacted directly or indirectly with genes related to these pathways (FIG. 3K). Collectively, these results suggest that HDAC3 controls LC development at least partially through direct or indirect regulation of genes essential for cell proliferation.

### Example 4. HDAC3 Deficiency does not Affect Cell Apoptosis During LC Development

The role of HDAC3 in the apoptosis of skin resident macrophages and LC precursors during embryonic development was studied by immunolabeling with annexin V and sorting cells by flow cytometry.

### Methods

Control and HDAC3-deficient skin and epidermal cell suspensions were obtained from HDAC3fl/fl and Csf1ricreHDAC3fl/fl embryonic day 16.5 (E16.5) and embryonic day 18.5 (E18.5) mice as described in Example 1. Immunolabeling and flow cytometry was performed as described in Example 1.

### Results

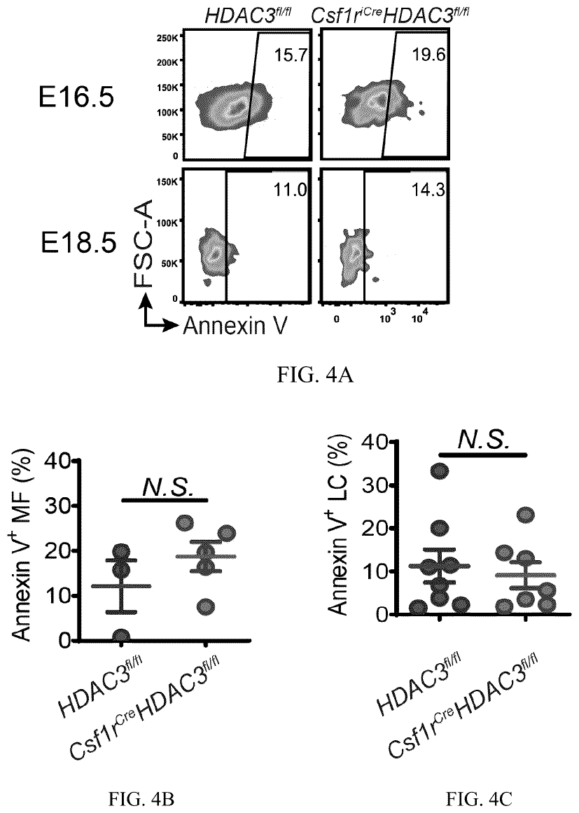

A flow cytometry analysis showed the frequencies of annexin V+ labeled skin resident macrophage (MF) and LC precursors are comparable in WT and HDAC3 cKO mice at E16.5 and E18.5, which indicates that HDAC3 deficiency does not affect cell apoptosis during LC development, and thus is unlikely to play a role in developmental apoptosis (FIGS. 4A, 4B, and 4C).

### Example 5. HDAC3 is Required for the Survival of Postnatal Epidermal LCs

The role of HDAC3 in postnatal maintenance of LCs in the epidermis was studied by examining LC precursor biomarkers in HDAC3 deficient LCs. A conditional postnatal HDAC3 cKO was generated by crossing huLangerinCre transgenic mice with loxp-flanked Hdac3fl/fl transgenic mice. The human Langerin transgene is only expressed in LCs and not by other murine Langerin expressing cells (e.g., muLangerin+ dermal DCs). Thus, the huLangerinCreHdac3fl/fl double transgenic selectively reduces HDAC3 gene expression LCs.

Additional conditional knock-outs were made to determine whether HDAC3 regulates the maintenance of fully differentiated LCs in during postnatal development and adult mice at steady state.

### Methods

huLangerinCre and huLangerinCreER mouse strains were provided by Daniel H. Kaplan, and Hdac3fl/fl mice were provided by Scott W. Hiebert. The genotype of huLangerinCre mice were determined by PCR of genomic DNA using conventional protocols and the following PCR primer pairs: mutant, forward, SEQ ID NO: 33 (5′-GAG GCA AAT GAT TGG CAT TCT AC-3′); mutant, reverse, SEQ ID NO: 34 (5′-CTG GAA AAT TCA AGA AGA GCC T-3′); WT, forward, SEQ ID NO: 35 (5′-GTT GCA GCA AAT TCC AAC CC-3′); WT, reverse, SEQ ID NO:36 (5′-GGC CAG GAG AAA CTA CAA GTC A-3′). The mutant allele produced a 272-bp PCR product whereas the WT allele resulted in a 167-bp PCR product. The mouse strain Cd11cCre (Strain #008068), was purchased from Jackson Laboratory (Bar Harbor, ME). Hdac3fl/fl mice were genotyped as described in Example 1 and crossed with huLangerinCre mice and CD11cCre mice to produce CD11cCreHdac3fl/fl and huLangerinCreHdac3fl/fl double transgenic mice, respectively. The CD11cCreHdac3fl/fl double transgenic mice strongly express Cre during the first week after birth in LCs resulting in the deletion of HDAC3 when LCs undergo a burst of proliferation.

LC-specific tamoxifen inducible HDAC3 cKO mice (huLangerinCreERHdac3fl/fl) were made by crossing the Hdac3fl/fl transgenic mouse with a transgenic mouse expressing Cre under the control of the CD11c promote, huLangerinCreER. Tamoxifen inducible Cre transgenic mice huLangerinCreER were purchased from purchased from Jackson Laboratory (Bar Harbor, ME). The huLangerinCreER mice were crossed with Hdac3fl/fl to generate the huLangerinCreERT2Hdac3fl/fl double transgenic mice. Mice were injected with tamoxifen to induce Cre-mediated excision of Hdac3. Deletion of Hdac3 was confirmed by qRT-PCR.

### Immunofluorescence

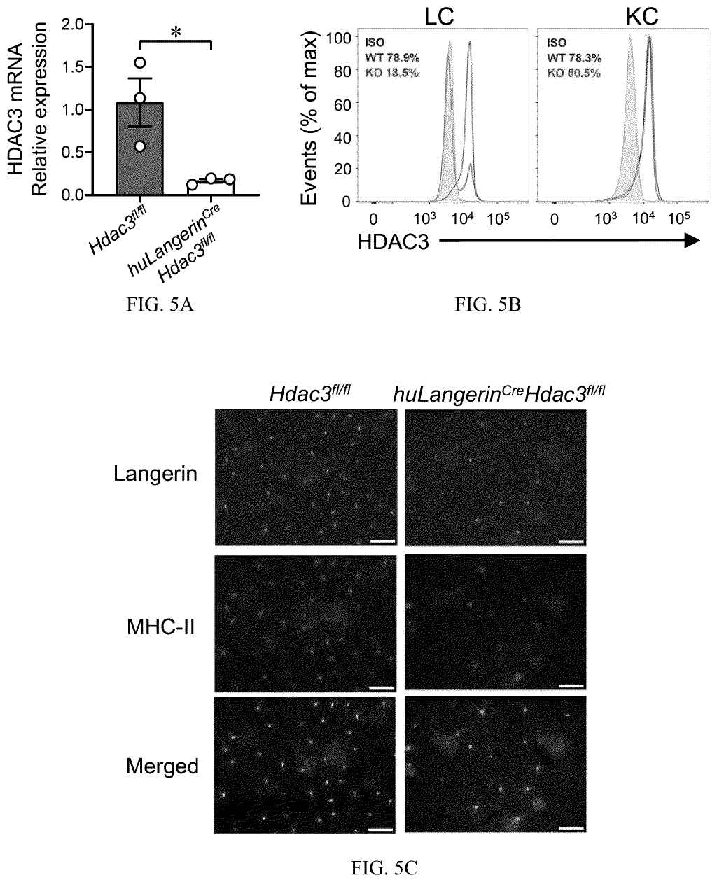

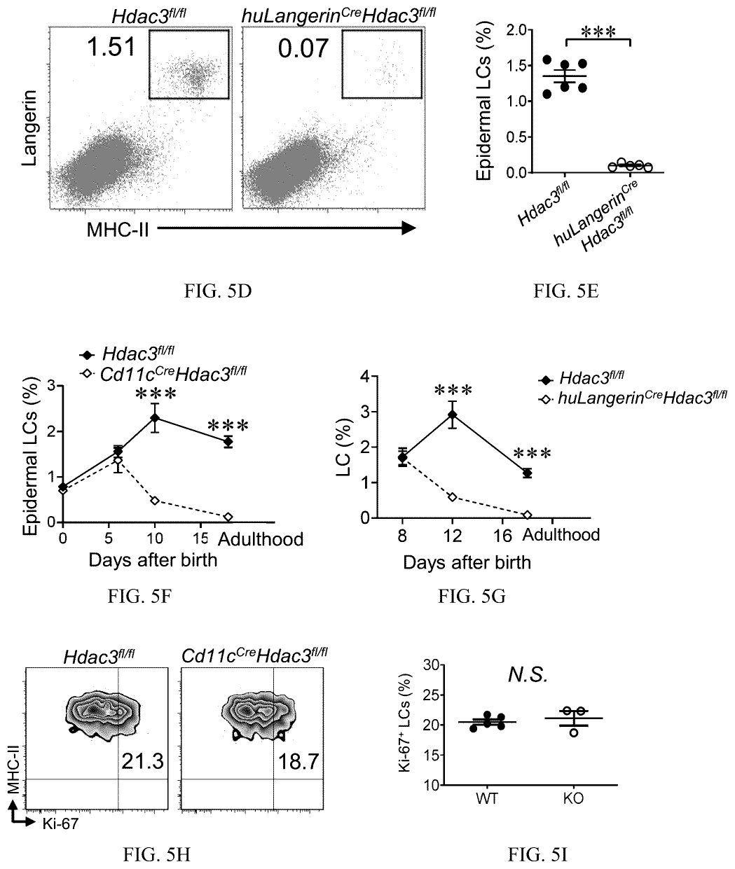

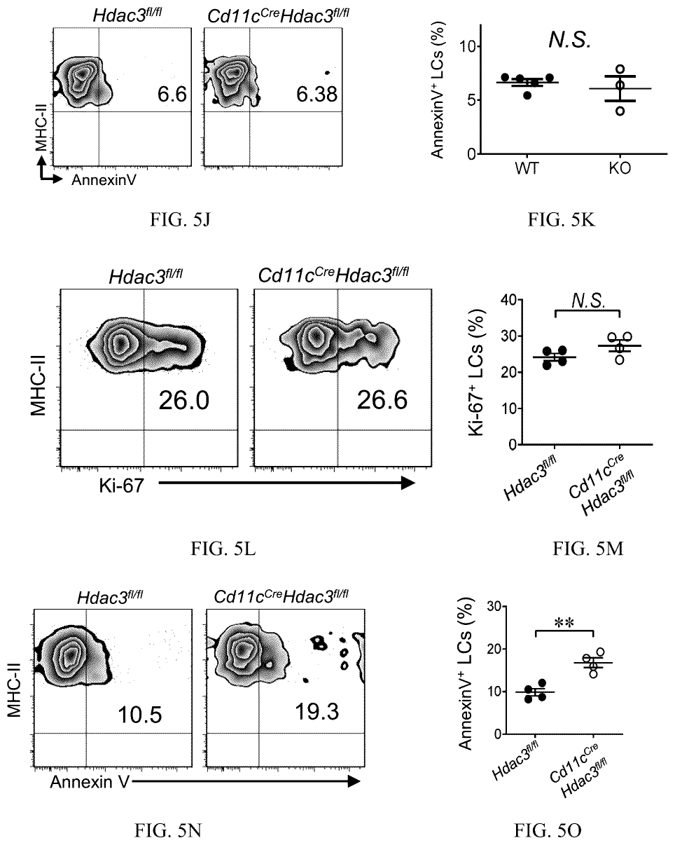

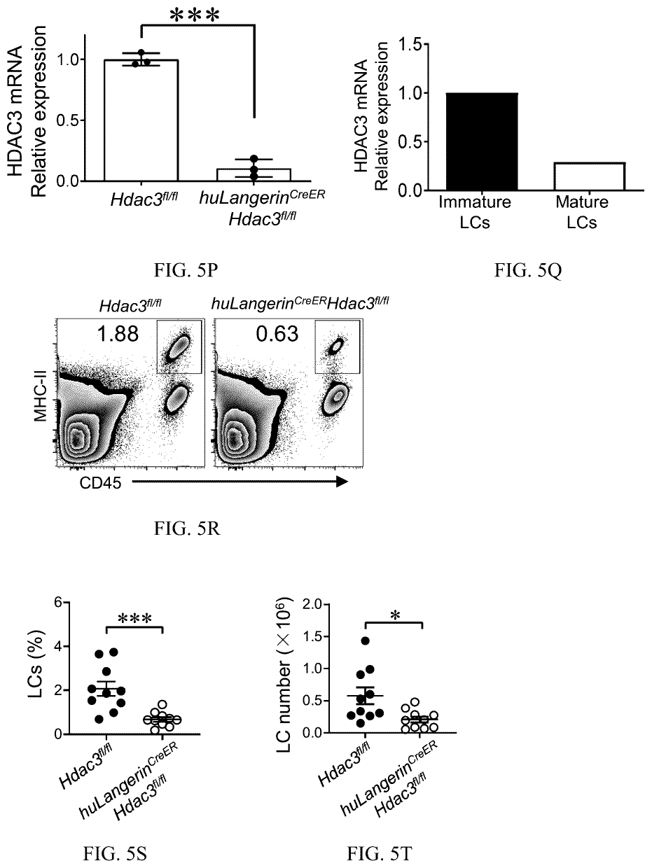

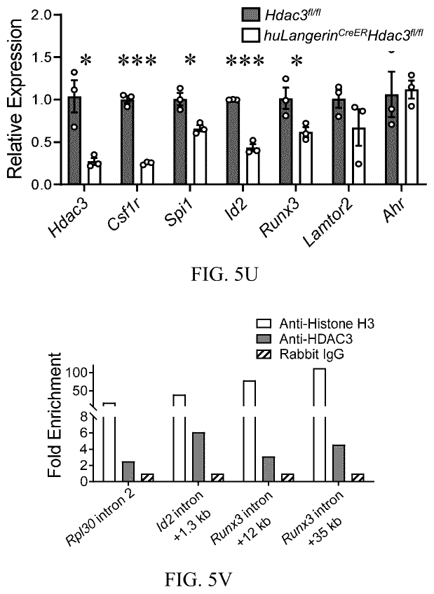

To visualize the density of epidermal LCs the epidermal sheets of mouse ear were gently peeled off the dermis after overnight treatment in PBS containing 20 mM EDTA (VWR Life Science, Radnor, PA). Separated epidermal sheets were fixed with cold acetone at −20° C. for 15 min, followed by blocking with PBS containing 3% bovine serum albumin (BSA) at room temperature (RT) for 15 min. The blocked sheets were incubated overnight with Alexa Fluor 488-labeled anti-langerin and PE-labeled anti-MHC-II antibodies at 4° C. in the dark, and then mounted under a coverslip with one drop of IMMU-MOUNT mounting medium (Thermo scientific). Specimens were viewed on a LEICA DMIRB microscope (Leica Microsystems, Buffalo Grove, IL) at 20× magnification (FIG. 5C).

### Tamoxifen Treatment

Tamoxifen (TAM, Sigma Aldrich) was dissolved in corn oil (Sigma Aldrich) containing 10% (vol/vol) ethanol (Thermo Fisher Scientific) and was intraperitoneally administered at 50 mg/kg of mouse weight for 5 consecutive days. For ex vivo LC function analysis mice were analyzed or further treated on day 9 after the last dose of TAM treatment. The deletion (>90%) of HDAC3 mRNAs in the epidermal LCs of tamoxifen treated huLangerinCreERHdac3fl/fl mice was confirmed by qRT-PCR.

### Results

The HDAC3 mRNA expression was robustly decreased (>80%) in the LCs from huLangerinCreHdac3 fl/fl mice (FIG. 5A). Moreover, HDAC3 protein levels determined by flow cytometry sorting for CD45-MHC II—were dramatically reduced (>75%) in LCs but not in keratinocytes (KC) in 3-week old huLangerinCreHdac3 fl/fl mice (FIG. 5B). The results demonstrated that the Hdac3 gene was efficiently removed in a LC-specific fashion. Epidermal sheets were labeled by immunofluorescence antibodies. The density of LCs (MHC II+langerin+) was greatly reduced in the epidermal sheets from huLangerinCreHdac3 fl/fl mice compared to WT mice (FIG. 5C). Consistently, flow cytometric analyses of epidermal cells from huLangerinCreHdac3 fl/fl mice showed a nearly complete loss of LCs (FIGS. 5D and 5E). Therefore, the results suggest that HDAC3 appears to be required for homeostasis of epidermal LCs after birth until adulthood at steady state.

A flow cytometry analysis demonstrated that the frequency of MHC II+langerin+epidermal LCs was comparable between CD11cCreHdac3fl/fl KO and WT mice by P6 (FIG. 5F). As expected, the LC frequency increased robustly from P6 to P10 in the WT mice. However, in the CD11cCreHdac3fl/fl KO mice, the LC frequency was remarkably decreased in P10 and almost completely diminished (<0.1%) in adulthood (FIG. 5F), which was consistent with our observations in the huLangerinCreHdac3 fl/fl mice (FIGS. 5E and 5G). Together, these results indicate that HDAC3 is essential to the initial establishment of the LC compartment, especially during the rapid proliferation phase.

Given that minimal external input of LC precursors occurs after birth in steady state, the loss of LCs caused by HDAC3 deletion could be attributable to increased apoptosis. At postnatal day 6 there was no difference in the frequency of Ki-67+ LCs (FIGS. 5H and 5I) or annexin V+ labeled LCs between CD11cCreHdac3fl/fl KO mice and Hdac3fl/fl mice (FIGS. 5J and 5K).

At postnatal day 10 there was no difference in the frequency of Ki-67+ LCs (FIGS. 5L and 5M) between CD11cCreHdac3fl/fl KO mice and Hdac3fl/fl mice (FIGS. 5L and 5M). However, at postnatal day 10, the HDAC3 KO mice had a higher frequency of annexin V+ labeled LCs, than in Hdac3fl/fl mice (FIGS. 5N and 5O). These results suggest that HDAC3 deletion promotes the apoptosis of LCs, which may have partially contributed to the loss of LCs in HDAC3-deficient mice during the early stage after birth (P10).

Relative HDAC3 mRNA expression as measured by qRT-PCR was significantly reduced in MHCII+CD45+epidermal LCs from huLangerinCreERHdac3 fl/fl mice compared to Hdac3 fl/fl mice 10 days after tamoxifen injection (FIG. 5P). The results indicate that about 75% of epidermal LCs undergo Cre-mediated excision 10 days after TAM injection. Relative HDAC3 mRNA expression was also significantly reduced in MHCII+CD45+epidermal LCs from WT mice after culturing for 60 hrs (mature LCs) compared to freshly isolated (immature LCs) (FIG. 5Q). A flow cytometry analysis showed that the frequency and number of CD45+MHC-II epidermal LCs in huLangerinCreERHDAC3fl/fl mice were significantly lower than in WT mice on days 8-10 after TAM treatment (FIGS. 5R, 5S, and 5T).

Several key genes are required for LC homeostasis, including Csf1r (encoding macrophage colony-stimulating factor 1 receptor, M-CSFR), Spi1 (purine rich box-1, PU.1), Id2 (inhibitor of DNA binding 2), Runx3 (runt related transcription factor 3), Lamtor2 (late endosomal adaptor molecule p14) and Ahr (aryl hydrocarbon receptor). A qRT-PCR analysis confirmed the dramatic reduction in mRNA expression of Csf1r, Spi1, Id2, and Runx3 in the LCs from TAM-treated huLangerinCreERHdac3fl/fl mice compared to their WT counterparts (FIG. 5U). Interestingly, ChIP-qPCR analysis using XS-106 cells further revealed that HDAC3 binds to the intronic regions of ID2 (+1.3 kilobases (kb)) and Runx3 (+12 kb and +35 kb) from the transcription start sites (FIG. 5V), suggesting that ID2 and RUNX3 might be downstream targets of HDAC3 that regulate LC maintenance.

Collectively, these results suggest that HDAC3 is required for epidermal LC homeostasis during initial establishment of the LC network as well as steady-state self-renewal. In contrast to its role during embryonic development, HDAC3 regulates cell survival for LC maintenance after birth rather than cell proliferation, likely through direct regulation of two transcription factors ID2 and RUNX3 that are essential for LC homeostasis.

### Example 6. HDAC3 Deficiency Affects Long-Term LC Repopulation but does not Impede Short-Term LC Generation

The role of HDAC3 during LC repopulation was examined under inflammatory conditions. During inflammation, short-term LCs (MHC II+langerinlow/neg) derived from circulating Gr-1hi monocytes and long-term LCs (MHC II+langerin+) of BM origin transiently or persistently reconstitute the LC compartment, respectively. In contrast to steady-state LC development and/or maintenance, which depends on PU.1, ID2 and RUNX3, the generation of short-term LCs is ID2 independent, while the generation of BM-derived LCs depends on PU.1-mediated RUNX3 expression. Given the significant loss of ID2 and RUNX3 mRNA expression in HDAC3-deficient LCs, as well as direct binding of HDAC3 to ID2 and Runx3 enhancer regions (FIGS. 5U and 5V), HDAC3 may play a role in repopulating LC precursors under inflammatory conditions.

### Methods

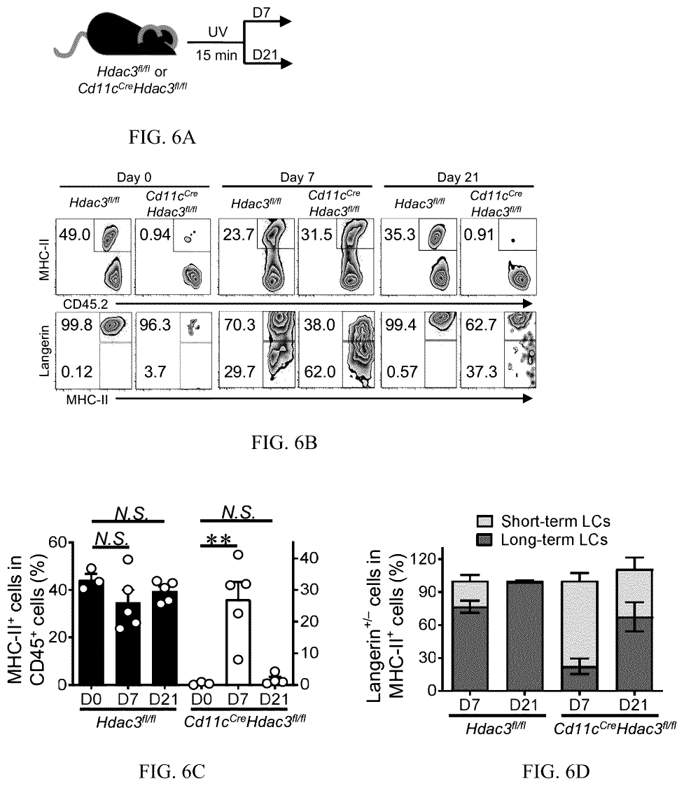

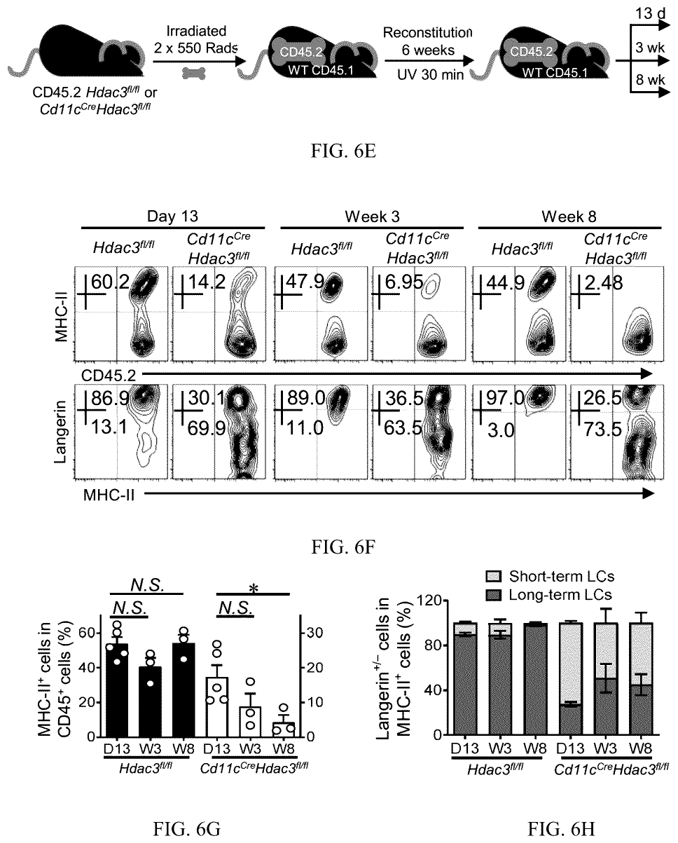

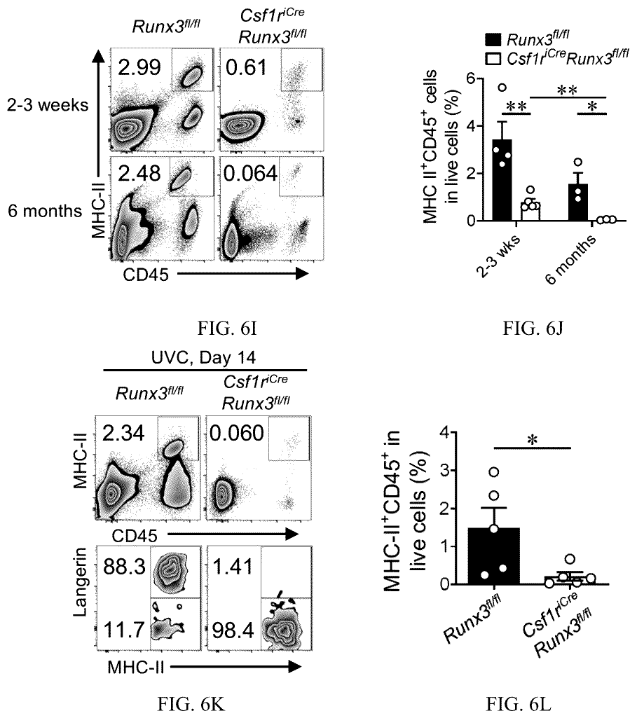

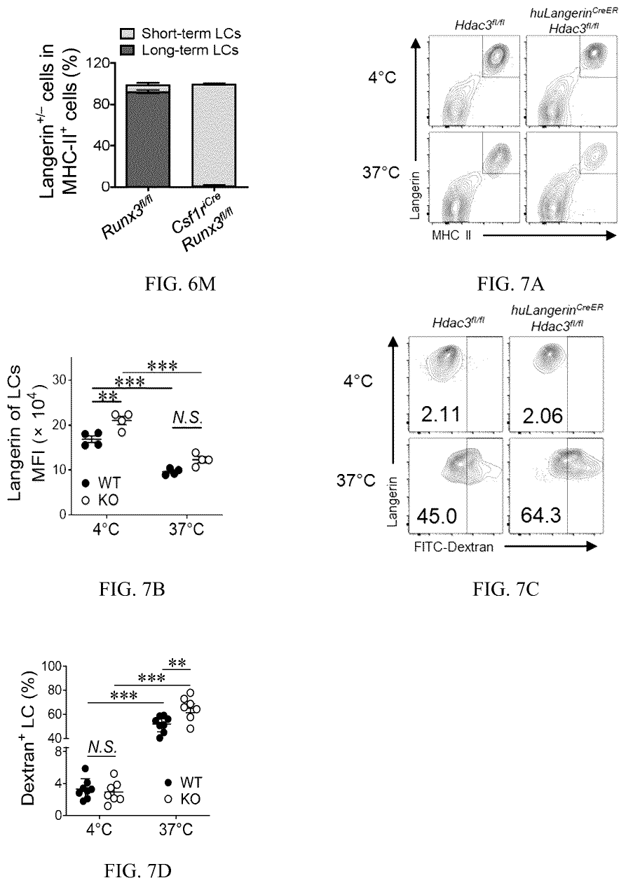

The role of HDAC3 in repopulating LC precursors during the inflammatory skin condition produced by UV radiation was examined in the dorsal skin from Hdac3fl/fl WT and Cd11cCreHDAC3fl/fl KO mice. Mice were ultraviolet-C (UVC) irradiated for 15 minutes and LC repopulation evaluated on days 7 and 21 after UVC exposure (see schematic, FIG. 6A). Immunolabeling and cell sorting by flow cytometry was previously described in Example 1 methods.

### Skin Inflammation

LC-depleting skin inflammation was induced on shaved mouse dorsal skin by exposure to ultraviolet light (UV; wavelength: 254 nm; voltage: 8 W; source distance: 38 cm) for 15 or 30 min. The timepoints for evaluating epidermal LC replenishment depended on different UV exposure times (UV exposure for 15 min was evaluated 1 and 3 weeks; UV exposure for 30 min was evaluated at 3 and 6 weeks).

### Bone Marrow Chimeras

To exclude the confounding factor of incomplete LC depletion by UVC exposure and to further evaluate the role of HDAC3 during long-term LC repopulation, irradiated B6.SJL mice (CD45.1+) were reconstituted with BM cells from Hdac3fl/fl (WT) or Cd11cCreHDAC3fl/fl KO mice (both CD45.2+). Six weeks after reconstitution, the recipient mice received UVC treatment for 30 min to deplete host LCs. B6.SJL (CD45.1+) mice were lethally irradiated with 550 Rads for 2 times. Donor BM cells were harvested from age- and sex-matched CD45.2+Hdac3fl/fl and Cd11cCreHDAC3fl/fl mice by flushing bones with a syringe containing sterile RPMI 1640 medium. After the lysis of erythrocytes, 100 μl of PBS containing approximately 10⁷ BM cells from either HDAC3fl/fl or Cd11cCreHDAC3fl/fl mice was retroorbitally administered into each irradiated recipient mouse. The recipient mice received further treatment until 6 weeks after reconstitution. The B6.SJL mouse strain (#002014) was purchased from Jackson Laboratory (Bar Harbor, ME).

HDAC3 KOs have reduced RUNX3 expression (FIG. 5U). To determine whether RUNX3 is required for long-term LC generation during in vivo inflammation, RUNX3 KO mice (Csf1riCreRunx3fl/fl) were generated wherein myeloid cells derived from both embryonic and BM progenitor cells are RUNX3 deficient. The Runx3fl/fl mouse strain (#008773) was purchased from the Jackson Laboratory (Bar Harbor, ME). Runx3fl/fl mice were genotyped using the following PCR primer pair: forward, SEQ ID NO: 45 (5′-TAT CCC TCT CTG GGC CTT CT-3′); reverse, SEQ ID NO: 46 (5′-GGA AAC TGA GTC CAG CCA AG-3′). The floxed allele produced a 325-bp PCR product whereas the wild-type (WT) allele resulted in a 199-bp PCR product.

### Results

As expected, a flow cytometry analysis showed around 40% of CD45+ cells in the epidermis were LCs (MHC II+) prior to UVC treatment (Day 0) in WT mice, while very few LCs existed in the Cd11cCreHDAC3fl/fl KO mice (FIG. 6B). After UVC exposure, the frequency of MHC II+ cells remained unaltered in the WT mice (FIGS. 6B, 6C). To the contrary, Cd11cCreHDAC3fl/fl KO mice displayed a significant increase in the MHC II+ cell ratio 7 days after UVC exposure, although this recruitment of MHC II+ cells was only transient as the LC frequency dropped to the steady-state level by 21 days (FIGS. 6B, 6C).

Short-term LCs were distinguished from long-term LCs in these mice by langerin expression in MHC II+ cells. In contrast to the steady state, where nearly all LCs in HDAC3fl/fl (WT) and Cd11cCreHDAC3fl/fl KO mice highly expressed langerin, a significant number of MHC II+langerinlow/neg short-term LCs emerged in the inflamed skin in both groups (FIGS. 6B, 6D). In WT mice, the frequency of short-term LCs increased to ˜30% and MHC II+langerin+ long-term LCs decreased to ˜70% 7 days after UVC treatment; although the short-term LCs were eventually replaced by long-term LCs. In Cd11cCreHDAC3fl/fl KO mice, the majority (>60%) of temporarily replenished MHC-II+ cells were short-term LCs on day 7 post-UVC treatment, which were still detectable by 21 days. These results suggest that HDAC3 is dispensable for short-term LC repopulation.

Six weeks after reconstituting irradiated B6.SJL mice (CD45.1+) with BM cells from Hdac3fl/fl (WT) or Cd11cCreHDAC3fl/fl KO mice, the recipient mice received UVC treatment for 30 min to deplete host LCs. Subsequently, the recruitment of donor cells into the epidermis was monitored by flow cytometry for 13 days, 3 weeks and 8 weeks after UVC treatment (treatment schematic, FIG. 6E). Analyses of spleen and LN cells showed a comparable degree of chimerism, regardless of the donor genotypes (data not shown). The B6.SJL recipient mice reconstituted with the BM cells from WT mice underwent a typical sequential repopulation by short-term and long-term LCs during the 8 weeks after UVC treatment (FIGS. 6F-6H). In contrast, the recipient mice reconstituted with the BM cells from the Cd11cCreHDAC3fl/fl KO mice had substantially fewer repopulated donor MHC-II+ cells, which contained a higher ratio of short-term LCs that eventually almost disappeared (<5%) around 8 weeks after UVC treatment. Taken together, these data demonstrate that lack of HDAC3 prevents the repopulation of long-term LCs but does not impede short-term LC generation.

RUNX3-null mice completely lack LCs in skin epidermis at steady state and RUNX3 drives LC differentiation from BM precursors in vitro. As expected, LC frequency was dramatic reduced in mice lacking RUNX3 compared to WT in young mice (2- to 3-weeks old), and the loss of LCs was even more severe in RUNX3-deficient mature adult mice (6-months old) (FIGS. 6I, 6J). Next, the long-term LC repopulation of LCs was examined in Csf1riCreRunx3fl/fl mice during UVC-induced skin inflammation. Two weeks after UVC exposure, very few MHC II+CD45+ cells were detected in the epidermis of Csf1riCreRunx3fl/fl KO mice, with the frequency of these cells significantly lower in the RUNX3-deficient mice than WT mice (FIGS. 6K, 6L). Moreover, in contrast to robust emergence of MHC II+langerin+ long-term LCs in the WT mice after UVC treatment, the vast of majority of the MHC II+ LCs lacked langerin expression in the RUNX3-deficient mice, indicating that these cells were short-term LCs derived from circulating monocytes (FIGS. 6K, 6M). Collectively, these results suggest that RUNX3 is critical for long-term LC generation from BM during mouse skin inflammation. The data indicates that HDAC3 could bind directly to Runx3 enhancers to regulate Runx3 transcription in LC precursors (FIG. 5U), which implies that HDAC3 controls long-term LC repopulation possibly through direct regulation of RUNX3.

### Example 7. HDAC3 Negatively Regulates LC Phagocytosis and Maturation

The role of HDAC3 in the LC migration, phagocytosis, and LC-mediated T cell immunity was studied in ex vivo cell culture from cells isolated from adult tamoxifen treated huLangerinCreERHdac3fl/fl mice.

### Methods

The formulation of TAM and the administration of TAM in mice was performed as described in Example 5 methods.

### Cell Culture

Murine XS-106 epidermal DC line and NS47 fibroblast cell line were a generous gift from Dr. Akira Takashima (University of Toledo). XS-106 cells were cultured in RPMI 1640 medium (Gibco//Thermo Fisher Scientific, Waltham, MA) supplemented with 2 mM L-glutamine, 10% fetal bovine serum (FBS, Hyclone, San Angelo, TX), 2.5 ng/ml murine recombinant granulocyte-macrophage colony stimulating factor (BioLegend, San Diego, CA), 5% NS47 fibroblast supernatant, and 1% penicillin-streptomycin-amphotericin B (Sigma Aldrich, St. Louis, MO). NS47 fibroblasts were maintained in RPMI 1640 medium supplemented with 2 mM L-glutamine, 10% FBS and 1% penicillin-streptomycin-amphotericin B, and subcultured (1:4-1:6 split ratio) using 0.25% trypsin-EDTA (Corning). Cells were maintained in a humidified 5% CO2 atmosphere at 37° C. and subcultured by harvesting floating cells from confluent T-75 culture flasks (Corning Incorporated, Corning NY). NS47 fibroblast supernatant was obtained by maintaining confluent cells in T-75 flasks in a minimal volume of culture medium for 4 days. The supernatant was centrifuged and stored at −80° C. prior to use.

### Adult Tissue Preparation for Flow Cytometry

Adult mice were sacrificed by CO₂ exposure. Mouse skin was dissociated and incubated at 37° C. for 1 hour in phosphate-buffered saline (PBS), Invitrogen, Carlsbad, CA) containing 0.5% dispase II (Gibco). Epidermal sheets were then separated from dermis, cut into small pieces and digested at 37° C. for 15 min in PBS containing 0.025% trypsin and 0.01% DNase I (Worthington Biochemical Corp., Lakewood, NJ). For LC maturation, phagocytosis, and co-culture experiments, epidermal tissues were digested at 37° C. for 1 hour in the RPMI 1640 medium containing 10% FBS and 0.010% DNase I.

### Ex Vivo Maturation

Freshly-separated epidermal cells were cultured in complete RPMI 1640 culture medium containing 10% FBS, 2 mM L-glutamine, 50 μM 2-mercaptoethanol, 0.15% sodium hydrogen carbonate, 1 mM sodium pyruvate, nonessential amino acids, 100 U/ml penicillin and 0.01% streptomycin at 37° C. with 5% CO₂ for 66-68 hrs. Cells were then stained with antibodies specific for MHC-II, CD45, CD80 and CD86 for the flow cytometric analysis.

### Skin-Draining Axillary and Inguinal

LNs were harvested, minced, and incubated at 37° C. for 15 min in PBS containing 0.01% DNase I. Spleen was collected and ground, and erythrocytes were lysed with 0.83% NH4Cl buffer.

### Phagocytosis Assay

Freshly isolated epidermal cell suspensions were cultured with 0.025% fluorescein isothiocyanate (FITC)-dextran (ThermoFisher Scientific, 40,000 MW) for 45 min at 37° C. or 4° C. After extensive washes with PBS, cells were stained with anti-CD45-APC and anti-MHC-II-PE antibodies. The percentage of FITC+ LCs (CD45.2+MHC-II+) was determined by flow cytometry.

### Results

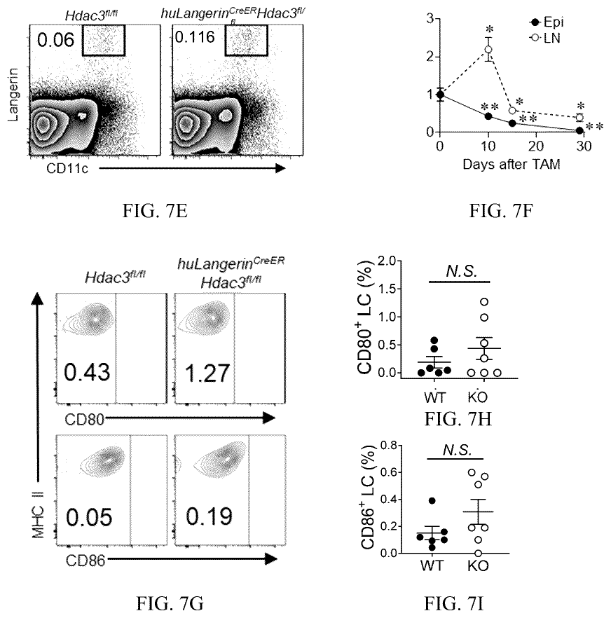

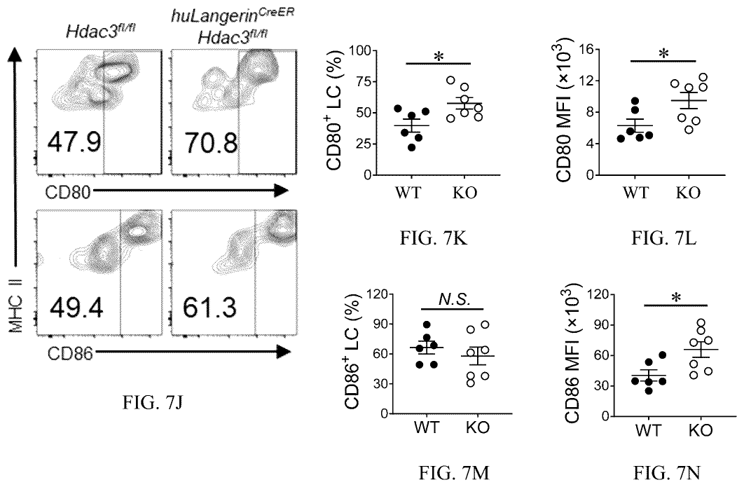

Residing at the outermost skin layer, LCs efficiently ingest and process environmental and self-antigens, which is vital to the induction of immune defense and tolerance. To assess the role of HDAC3 in the phagocytic capacity of LCs, freshly-isolated epidermal cells from Hdac3fl/fl (WT) and huLangerinCreERHDAC3fl/fl KO mice were isolated 9-days post TAM treatment and incubated with FITC-dextran for 45 min at 4° C. (as control) or 37° C. The surface expression of langerin in MHC II+epidermal LCs indicates that the innate immune receptor langerin can interact directly with viruses, bacteria, and fungi, and langerin-mediated LC phagocytosis is critical for host defense against microbial pathogens. Langerin expression in epidermal LCs was markedly increased in TAM-treated huLangerinCreERHDAC3fl/fl KO mice compared to WT mice at 4° C. and 37° C. (FIGS. 7A, 7B).

Interestingly, a dramatic decrease in langerin surface expression in WT and KO LCs occurred at 37° C. compared to 4° C., indicating that langerin internalization may facilitate LC antigen uptake. Additionally, the frequencies of FITC-dextran+ LCs from huLangerinCreERHDAC3fl/fl KO mice were significantly higher than WT mice when incubated at 37° C. (FIGS. 7C and 7D). Taken together, these results indicate that HDAC3 plays an inhibitory role in the phagocytotic capacity of epidermal LCs.

As shown in FIG. 5S, the TAM inducible deletion of HDAC3 in huLangerinCreERHDAC3fl/fl mice led to a significant reduction in the number of LCs in the epidermis. In parallel, the number of migrated LCs (CD207+CD11c+) derived from skin-draining lymph nodes isolated from huLangerinCreERHDAC3fl/fl mice increased 10 days after tamoxifen injection (FIGS. 7E, 7F).

The peak (˜2 fold) of LC migration was found on day 10 after TAM treatment, followed by a concomitant decrease of LCs in both epidermis and skin-draining LNs on days 15 and 30 post-TAM treatment (FIGS. 7E, 7F). Thus, HDAC3 deficiency resulted in a spontaneous migration of LCs from the epidermis into the LNs. It is known that LC migration is usually coupled with maturation status.

During steady state, LCs locating to the epidermis are immature and barely express co-stimulatory molecules (e.g., CD80 and CD86). Upon exposure to inflammatory stimuli or during ex vivo culture, LCs undergo maturation by upregulating the expression of these co-stimulatory molecules and emigrate from the skin to the LNs, which is considered critical for T cell priming in response to inflammation. Thus, the spontaneous migration of HDAC3-deficient LCs may have resulted from the enhanced maturation of epidermal LCs in the absence of HDAC3.

HDAC3 mRNA expression in LCs from WT adult mice before and after ex vivo culture was over 70% lower in cultured mature LCs than in freshly isolated immature LCs (see example 5, FIG. 5Q), suggesting that the low expression of HDAC3 is required for LC maturation. To determine if HDAC3 regulates LC maturation the surface expression of co-stimulatory molecules were identified by immunolabeling and flow cytometry in immature and mature LCs, from WT and huLangerinCreERHDAC3fl/fl KO mice, before and after ex vivo culture. As expected, freshly isolated epidermal LCs from both Hdac3fl/fl WT and huLangerinCreERHDAC3fl/fl KO mice after TAM treatment expressed extremely low levels (<2.0%) of CD80 and CD86 (FIGS. 7G, 7H, and 7I). On the other hand, after 66-68 hours of ex vivo culture, a large number of CD80+(20%-50%) and CD86+(40%-90%) LCs were detected in the WT cell culture (FIG. 7J). Interestingly, the expression of both CD80 and CD86 were significantly increased in the cultured LCs from TAM-treated huLangerinCreERHDAC3fl/fl KO mice (FIGS. 7J, 7K, 7M, and 7N). Consistently, the frequencies of CD80+ and CD86+ LCs were increased in the huLangerinCreERHDAC3fl/fl mice in which HDAC3 was constitutively deleted in LCs. The results indicate that HDAC3 deficiency enhances maturation of LCs, leading to their spontaneous migration from the epidermis to the LNs. According to these results, treating LCH and LCS patients with an HDAC3 inhibitor may enhance LC maturation and migration to the LNs, as well as inhibit the phagocytotic capacity of LCs.

### Example 8. HDAC3 Inhibitor Reduces DC Frequency in BRAFV600E Mutant Mice

Langerhans cell histiocytosis (LCH) is a pediatric inflammatory myeloid neoplasm wherein myeloid lineage cells accumulate in tissues throughout the body. LCH was named for the presence of mutation-bearing myeloid cells that irregularly express langerin (CD207) and CD1a, molecules known to be expressed on Langerhans cells.

The most common mutation in LCH is BRAFV600E, which leads to constitutive activation of RAF-MEK-ERK signaling in the absence of mitogens. Constitutive activation of this signaling cascade causes the pathophysiologic changes observed in myeloid cells that drive LCH pathology. The BRAFV600E mutation causes mTOR-induced senescence-associated secretory phenotype, reduced apoptosis, abrogated tissue migration, and dysregulated hematopoiesis.

BMDC cultures were generated from mice bearing this mutation in myeloid lineage cells, which is a valuable tool for identifying drugs that can inhibit BRAFV600E-bearing DCs presumably LCH cells or LCH precursor cells

### Methods

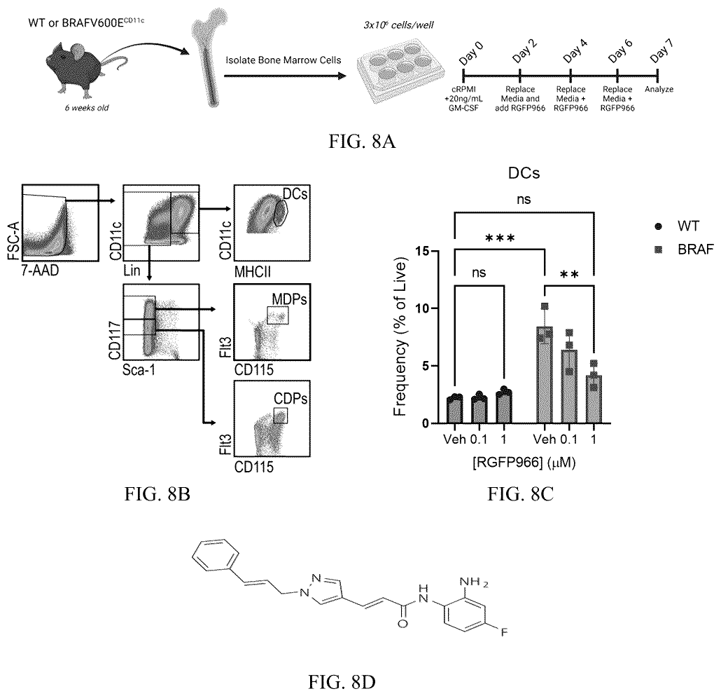

BMDCs are generated by isolating bone-marrow from mice and culturing the bone marrow in complete RPMI (cRPMI) supplemented with 20 ng/mL of granulocyte-macrophage colony-stimulating factor (GM-CSF) for 6 days. The small-molecule HDAC3 inhibitor, RGFP966 (FIG. 9D), was tested for its ability to attenuate the development of BMDC-derived LCH cells. RGFP966 has an IC50 of 0.08 μM in cell-free assays and has been used at 10 μM to attenuate the growth of cutaneous T cell lymphoma cell lines. BMDC cultures derived from CD11ccreBRAFV600Eca/ca or ca/+ (BRAFV600ECD11c) and wild type littermate controls were treated with RGFP966 at 0.1, 1.0 and 10 μM starting on day 2 (see methods schematic, FIG. 8A). BMDCs were immunolabeled for dendritic cell surface receptors and analyzed by flow cytometry on day 7.

### Results

As expected, the cultures showed increased BMDC frequency from BRAFV600E mice compared to the BMDC frequency from WT mice (FIGS. 8B, 8C). This is consistent with the myeloid skewing observed in vivo. RGFP966 had no effect on WT BMDC at doses 0.1 μM and 1.0 μM (FIG. 8C). However, in DCs from BRAFV600ECD11c-derived BMDC cultures, the addition of 1 μM RGFP966 led to a significant decrease in the frequency of DCs compared to WT mice (FIG. 8C), while there was no significant difference in the frequency of DCs between WT and BRAF mice. Thus, the HDAC3 inhibitor has a more pronounced effect reducing DC frequency in cells isolated from mice with the BRAFV600E mutation compared to wild type mice. Since the BRAFV600E mutation leads to clonal expansion of bearing myeloid progenitors, treatment with HDAC3 inhibitors could preferentially affect LCH cells over unaffected DCs.

In conclusion, the data in the disclosed examples demonstrates that HDAC3 plays a critical role as a key epigenetic regulator to control embryonic development, maintenance, and function of epidermal LCs. Importantly, the disclosure supports the use of HDAC3 inhibitors as a therapeutic treatment for the LC proliferative diseases LCH and LSC.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of preventing, treating, or ameliorating the symptoms of Langerhans Cell Histiocytosis (LCH) or Langerhans Cell Sarcoma (LCS) in a subject, comprising administering a therapeutically effective amount of a HDAC3 inhibitor to the subject in need thereof.

2. The method of claim 1, wherein the HDAC3 inhibitor comprises: Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, UF010, Tucidinostat (Chidamide), Citarinostat (ACY-241), Domatinostat (4SC-202), or HPOB.

3. The method of claim 1, wherein the HDAC3 inhibitor is a selective HDAC3 inhibitor.

4. The method of claim 3, wherein the selective HDAC3 inhibitor is ((2E)-N-(2-Amino-4-fluorophenyl)-3-[(2E)-1-(3-phenyl-2-propen-1-yl)-1H-pyrazol-4-yl]-2-propenamide, (E)-N-(2-amino-4-fluorophenyl)-3-(1-cinnamyl-1H-pyrazol-4-yl)acrylamide (RGFP966) having the structure:

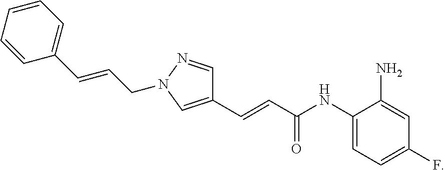 [SMILES: Nc1cc(F)ccc1NC(=O)/C=C/c1cnn(C/C=C/c2ccccc2)c1]

5. The method of claim 1, wherein the subject is a human pediatric subject less than 18 years of age.

6. The method of claim 5, wherein the subject is less than 16 years of age, less than 14 years of age, less than 12 years of age, less than 10 years of age, less than 8 years of age, less than 6 years of age, less than 4 years of age, or less than 2 years of age.

7. The method of claim 1, wherein LCH cells comprise at least a portion of the LCH cells or LCH precursor cells having a BRAFV600E mutation.

8. The method of claim 1, further comprising administering an effective amount of a combination comprising an HDAC3 inhibitor and a secondary active agent, which may be dosed to the subject concomitantly with, prior to, or sequentially to, the administration of the HDAC3 inhibitor.

9. The method of claim 8, wherein the secondary active agent comprises one or more of: a MAPK inhibitor, a chemotherapeutic agent or an immunomodulating agent.

10. The method according to claim 9, wherein the MAPK inhibitor comprises: dabrafenib, encorafenib, trametinib, tovorafenib, vemurafenib, PLX4720, GSK2118436 (GSK436), cobimetinib, RAF265 (also called CHIR-265, sorafenib, Tipifarnib, U0126 (EMD Biosciences (662005)), PD184352 (Axon Medchem (1368)), SB203580 (EMD Biosciences (55389) or combinations thereof.

11. The method according to claim 10, wherein the HDAC 3 inhibitor is selected from: Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, UF010, Tucidinostat (Chidamide), Citarinostat (ACY-241), Domatinostat (4SC-202), or HPOB, and the MAPK inhibitor is selected from: dabrafenib, encorafenib, trametinib, tovorafenib, vemurafenib, PLX4720, GSK2118436 (GSK436), cobimetinib, RAF265 (also called CHIR-265, sorafenib, Tipifarnib, U0126 (EMD Biosciences (662005)), PD184352 (Axon Medchem (1368)), SB203580 (EMD Biosciences (55389) or combinations thereof.

12. The method according to claim 10, wherein the combination comprises a selective HDAC3 inhibitor.

13. The method according to claim 12, wherein the combination comprises RGFP966 and a MAPK inhibitor selected from: dabrafenib, encorafenib, trametinib, tovorafenib, vemurafenib, PLX4720, GSK2118436 (GSK436), cobimetinib, RAF265 (also called CHIR-265, sorafenib, Tipifarnib, U0126 (EMD Biosciences (662005)), PD184352 (Axon Medchem (1368)), SB203580 (EMD Biosciences (55389) or combinations thereof.

14. The method according to claim 13, wherein the combination comprises RGFP966, and the MAPK inhibitor comprises one or more of: dabrafenib, trametinib, vemurafenib, and PLX4720.

15. The method according to claim 9, wherein secondary active agent is a chemotherapeutic agent selected from: selected from: cisplatin, carboplatin, 5-fluorouracil, cyclophosphamide, oncovin, vincristine, prednisone, or rituximab, mechlorethamine, cyclophosphamide, ifosfamide, melphalan, chlorambucil, carmustine, lomustine, semustine, thriethylenemelamine, triethylene thiophosphoramide, hexamethylmelamine altretamine, busulfan, triazines dacarbazine, methotrexate, trimetrexate, fluorodeoxyuridine, gemcitabine, cytosine arabinoside, 5-azacytidine, 2,2′-difluorodeoxycytidine, 6-mercaptopurine, 6-thioguanine, azathioprine, 2′-deoxycoformycin, erythrohydroxynonyladenine, fludarabine phosphate, 2-chlorodeoxyadenosine, camptothecin, topotecan, irinotecan, paclitaxel, vinblastine, vincristine, vinorelbine, docetaxel, estramustine, estramustine phosphate, etoposide, teniposide, mitoxantrone, mitotane, aminoglutethimide or combinations thereof.

16. The method of claim 15, wherein the HDAC3 inhibitor is RGFP966, and the chemotherapeutic agents comprises: cisplatin, carboplatin, 5-fluorouracil, cyclophosphamide, oncovin, vincristine, prednisone, or rituximab, mechlorethamine, ifosfamide, melphalan, chlorambucil, carmustine, lomustine, semustine, thriethylenemelamine, triethylene thiophosphoramide, hexamethylmelamine altretamine, busulfan, triazines dacarbazine, methotrexate, trimetrexate, fluorodeoxyuridine, gemcitabine, cytosine arabinoside, 5-azacytidine, 2,2′-difluorodeoxycytidine, 6-mercaptopurine, 6-thioguanine, azathioprine, 2′-deoxycoformycin, erythrohydroxynonyladenine, fludarabine phosphate, 2-chlorodeoxyadenosine, camptothecin, topotecan, irinotecan, paclitaxel, vinblastine, vincristine, vinorelbine, docetaxel, estramustine, estramustine phosphate, etoposide, teniposide, mitoxantrone, mitotane, aminoglutethimide or combinations thereof.

17. Use of a HDAC3 inhibitor for the manufacture of a medicament for preventing or treating LCH or LCS in a subject, comprising administering an effective or therapeutically effective dose to the subject that results in the prevention or treatment of LCH or LCS.

18. Use of a HDAC3 inhibitor for the prevention or treatment of LCH or LCS, comprising administering an effective or therapeutically effective dose to the subject that results in the prevention or treatment of LCH or LCS.

19. The use of claim 17, wherein the HDAC3 inhibitor comprises: Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, UF010, Tucidinostat (Chidamide), Citarinostat (ACY-241), Domatinostat (4SC-202), or HPOB.

20. The use of claim 17, wherein the HDAC3 inhibitor is a selective HDAC3 inhibitor.

21. The use of claim 17, wherein the HDAC3 inhibitor is ((2E)-N-(2-Amino-4-fluorophenyl)-3-[(2E)-1-(3-phenyl-2-propen-1-yl)-1H-pyrazol-4-yl]-2-propenamide, (E)-N-(2-amino-4-fluorophenyl)-3-(1-cinnamyl-1H-pyrazol-4-yl)acrylamide (RGFP966) having the structure:

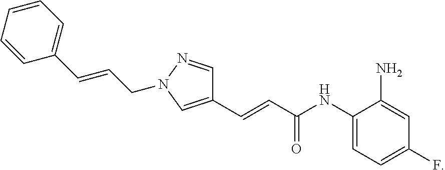 [SMILES: Nc1cc(F)ccc1NC(=O)/C=C/c1cnn(C/C=C/c2ccccc2)c1]

22. The use of claim 18, wherein the HDAC3 inhibitor comprises: Mocetinostat (MGCD0103), CUDC-101, Quisinostat (JNJ-26481585) 2HCI, Pracinostat (SB939), Droxinostat, Abexinostat (PCI-24781), Entinostat, Fimepinostat (CUDC-907), RG2833 (RGFP109), RGFP966, BRD3308, SR-4370, TC-H 106, UF010, Tucidinostat (Chidamide), Citarinostat (ACY-241), Domatinostat (4SC-202), or HPOB.

23. The use of claim 18, wherein the HDAC3 inhibitor is a selective HDAC3 inhibitor.

24. The use of claim 18, wherein the HDAC3 inhibitor is ((2E)-N-(2-Amino-4-fluorophenyl)-3-[(2E)-1-(3-phenyl-2-propen-1-yl)-1H-pyrazol-4-yl]-2-propenamide, (E)-N-(2-amino-4-fluorophenyl)-3-(1-cinnamyl-1H-pyrazol-4-yl)acrylamide (RGFP966) having the structure: 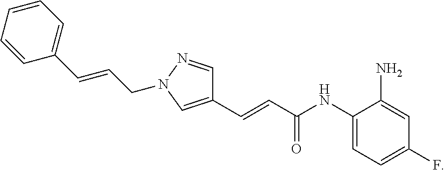 [SMILES: Nc1cc(F)ccc1NC(=O)/C=C/c1cnn(C/C=C/c2ccccc2)c1]


SEQUENCE LISTING (76 sequences)

SEQ ID NO: 1 - DNA, 18 bp, synthetic construct
  misc_feature 1..18: note = Actb (NG_007992) gene-Forward (5-3) primer sequence
  source 1..18: mol_type = other DNA
caacgagcggttccgatg

SEQ ID NO: 2 - DNA, 20 bp, synthetic construct
  misc_feature 1..20: note = Actb (NG_007992) geneReverse (5-3) primer sequence
  source 1..20: mol_type = other DNA
gccacaggattccataccca

SEQ ID NO: 3 - DNA, 22 bp, synthetic construct
  misc_feature 1..22: note = CGCTGAAACATGAGCAAATTGG
  source 1..22: mol_type = other DNA
cgctgaaacatgagcaaattgg

SEQ ID NO: 4 - DNA, 24 bp, synthetic construct
  misc_feature 1..24: note = Ahr (NM_001621) gene-Reverse (5-3) primer sequence
  source 1..24: mol_type = other DNA
acagcttaggtgctgagtcacagg

SEQ ID NO: 5 - DNA, 20 bp, synthetic construct
  misc_feature 1..20: note = Anapc2 (NM_013366.4) gene-Forward (5-3) primer sequence
  source 1..20: mol_type = other DNA
cgcctctatggtcgtttctt

SEQ ID NO: 6 - DNA, 22 bp, synthetic construct
  misc_feature 1..22: note = Anapc2 (NM_013366.4) gene-Reverse (5-3) primer sequence
  source 1..22: mol_type = other DNA
gctatctagctcaccctctagt

SEQ ID NO: 7 - DNA, 22 bp, synthetic construct
  misc_feature 1..22: note = Ccna2 (NG_052974.2) gene-Forward (5-3) primer sequence
  source 1..22: mol_type = other DNA
cactgacacctcttgactatcc

SEQ ID NO: 8 - DNA, 21 bp, synthetic construct
  misc_feature 1..21: note = Ccna2 (NG_052974.2) gene-Reverse (5-3) primer sequence
  source 1..21: mol_type = other DNA
cgttcactggcttgtcttcta

SEQ ID NO: 9 - DNA, 22 bp, synthetic construct
  misc_feature 1..22: note = Ccnb2 (NM_004701) gene-Forward (5-3) primer sequence
  source 1..22: mol_type = other DNA
ctctgcaagatcgaggacatag

SEQ ID NO: 10 - DNA, 20 bp, synthetic construct
  misc_feature 1..20: note = Ccnb2 (NM_004701) geneReverse (5-3) primer sequence
  source 1..20: mol_type = other DNA
tgcctgaggtactggtagat

SEQ ID NO: 11 - DNA, 21 bp, synthetic construct
  misc_feature 1..21: note = Cdc25b (NG_029040) gene-Forward (5-3) primer sequence
  source 1..21: mol_type = other DNA
cacctctcggtctttgagttt

SEQ ID NO: 12 - DNA, 21 bp, synthetic construct
  misc_feature 1..21: note = Cdc25b (NG_029040) gene-Reverse (5-3) primer sequence
  source 1..21: mol_type = other DNA
tgtgcatggtctgtgtaagag

SEQ ID NO: 13 - DNA, 22 bp, synthetic construct
  misc_feature 1..22: note = Cdca3 (NM_031299.7) gene-Forward (5-3) primer sequence
  source 1..22: mol_type = other DNA
agcgaagtattggagacagaag

SEQ ID NO: 14 - DNA, 22 bp, synthetic construct
  misc_feature 1..22: note = Cdca3(NM_031299.7) gene-Reverse (5-3) primer sequence
  source 1..22: mol_type = other DNA
gaagacaggtccaggtcataaa

SEQ ID NO: 15 - DNA, 21 bp, synthetic construct
  misc_feature 1..21: note = Chek2 (NG_008150) gene-Forward (5-3) primer sequence
  source 1..21: mol_type = other DNA
cctgaaagaagccacctgtaa

SEQ ID NO: 16 - DNA, 22 bp, synthetic construct
  misc_feature 1..22: note = Chek2 (NG_008150) gene-Reverse (5-3) primer sequence
  source 1..22: mol_type = other DNA
gtcccgatgtatgatcccattt

SEQ ID NO: 17 - DNA, 22 bp, synthetic construct
  misc_feature 1..22: note = Csf1r (NG_012303) gene-Forward (5-3) primer sequence
  source 1..22: mol_type = other DNA
atccccctaaacagtgacttcc

SEQ ID NO: 18 - DNA, 18 bp, synthetic construct
  misc_feature 1..18: note = Csf1r (NG_012303) gene-Reverse (5-3) primer sequence
  source 1..18: mol_type = other DNA
ggtcagattattccagcc

SEQ ID NO: 19 - DNA, 22 bp, synthetic construct
  misc_feature 1..22: note = Foxm1 (NG_029590) gene-Forward (5-3) primer sequence
  source 1..22: mol_type = other DNA
ccaggctggaagaactctattc

SEQ ID NO: 20 - DNA, 20 bp, synthetic construct
  misc_feature 1..20: note = Foxm1 (NG_029590) gene-Reverse (5-3) primer sequence
  source 1..20: mol_type = other DNA
ttgggtgaatggtccagaag

SEQ ID NO: 21 - DNA, 20 bp, synthetic construct
  misc_feature 1..20: note = Gapdh (NG_007073) gene-Forward (5-3) primer sequence
  source 1..20: mol_type = other DNA
ggtgaaggtcggtgtgaacg

SEQ ID NO: 22 - DNA, 23 bp, synthetic construct
  misc_feature 1..23: note = Gapdh (NG_007073) gene-Reverse (5-3) primer sequence
  source 1..23: mol_type = other DNA
tgtagaccatgtagttgaggtca

SEQ ID NO: 23 - DNA, 20 bp, synthetic construct
  misc_feature 1..20: note = Hdac3 (NG_029678) gene-Forward (5-3) primer sequence
  source 1..20: mol_type = other DNA
agctggacacccaatgaaac

SEQ ID NO: 24 - DNA, 20 bp, synthetic construct
  misc_feature 1..20: note = Hdac3 (NG_029678) gene-Reverse (5-3) primer sequence
  source 1..20: mol_type = other DNA
tgttgctccttgcagagatg

SEQ ID NO: 25 - DNA, 20 bp, synthetic construct
  misc_feature 1..20: note = Id2 (NM_002166) gene-Forward (5-3) primer sequence
  source 1..20: mol_type = other DNA
aaaacagcctgtcggaccac

SEQ ID NO: 26 - DNA, 20 bp, synthetic construct
  misc_feature 1..20: note = Id2 (NM_002166) geneReverse (5-3) primer sequence
  source 1..20: mol_type = other DNA
ctgggcaccagttccttgag

SEQ ID NO: 27 - DNA, 18 bp, synthetic construct
  misc_feature 1..18: note = Kifc1 (NM_002263.4) gene-Forward (5-3) primer sequence
  source 1..18: mol_type = other DNA
tgcctttgcaccctttct

SEQ ID NO: 28 - DNA, 23 bp, synthetic construct
  misc_feature 1..23: note = Kifc1 (NM_002263.4) gene-Reverse (5-3) primer sequence
  source 1..23: mol_type = other DNA
ccttcagttctacgttcctcttc

SEQ ID NO: 29 - DNA, 21 bp, synthetic construct
  misc_feature 1..21: note = Kat2b (NM_003884.5) gene-Forward (5-3) primer sequence
  source 1..21: mol_type = other DNA
aggagacctccagcagataat

SEQ ID NO: 30 - DNA, 23 bp, synthetic construct
  misc_feature 1..23: note = Kat2b (NM_003884.5) gene-Reverse (5-3) primer sequence
  source 1..23: mol_type = other DNA
ccatctcttcctctgacacattc

SEQ ID NO: 31 - DNA, 20 bp, synthetic construct
  misc_feature 1..20: note = Lamtor2 (NG_009898) gene-Forward (5-3) primer sequence
  source 1..20: mol_type = other DNA
tctgggccgcgtatgatagg

SEQ ID NO: 32 - DNA, 24 bp, synthetic construct
  misc_feature 1..24: note = Lamtor2 (NG_009898) gene-Reverse (5-3) primer sequence
  source 1..24: mol_type = other DNA
cacgctgttatgatgctgctactt

SEQ ID NO: 33 - DNA, 21 bp, synthetic construct
  misc_feature 1..21: note = huLangerinCre (AJ242859) gene-Forward (5-3) primer sequence
  source 1..21: mol_type = other DNA
gaggcaaatgattggcattct

SEQ ID NO: 34 - DNA, 21 bp, synthetic construct
  misc_feature 1..21: note = huLangerinCre (AJ242859) gene-Reverse (5-3) primer sequence
  source 1..21: mol_type = other DNA
ctggaaaattcaagaagagcc

SEQ ID NO: 35 - DNA, 20 bp, synthetic construct
  misc_feature 1..20: note = Langerin-WT (AJ242859) gene-Forward (5-3) primer sequence
  source 1..20: mol_type = other DNA
gttgcagcaaattccaaccc

SEQ ID NO: 36 - DNA, 22 bp, synthetic construct
  misc_feature 1..22: note = Langerin-WT (AJ242859) gene-Reverse (5-3) primer sequence
  source 1..22: mol_type = other DNA
ggccaggagaaactacaagtca

SEQ ID NO: 37 - DNA, 22 bp, synthetic construct
  misc_feature 1..22: note = Mad2l1 (NM_002358) gene-Forward (5-3) primer sequence
  source 1..22: mol_type = other DNA
ggactcaccttgcttacaacta

SEQ ID NO: 38 - DNA, 20 bp, synthetic construct
  misc_feature 1..20: note = Mad2l1 (NM_002358) gene-Reverse (5-3) primer sequence
  source 1..20: mol_type = other DNA
gatgaccaccaccagtttct

SEQ ID NO: 39 - DNA, 20 bp, synthetic construct
  misc_feature 1..20: note = Nostrin (NM_001039724.4) gene-Forward (5-3) primer sequence
  source 1..20: mol_type = other DNA
gatgccaagagccagaaaga

SEQ ID NO: 40 - DNA, 22 bp, synthetic construct
  misc_feature 1..22: note = Nostrin (NM_001039724.4) gene-Reverse (5-3) primer sequence
  source 1..22: mol_type = other DNA
cactgacgacagtttgtaggag

SEQ ID NO: 41 - DNA, 20 bp, synthetic construct
  misc_feature 1..20: note = Plau (NG_011904) geneForward (5-3) primer sequence
  source 1..20: mol_type = other DNA
cgcacactgcttcattcaac

SEQ ID NO: 42 - DNA, 21 bp, synthetic construct
  misc_feature 1..21: note = Plau (NG_011904) gene-Reverse (5-3) primer sequence
  source 1..21: mol_type = other DNA
gctccacctcaaacttcatct

SEQ ID NO: 43 - DNA, 21 bp, synthetic construct
  misc_feature 1..21: note = Rgcc (NM_014059) gene-Forward (5-3) primer sequence
  source 1..21: mol_type = other DNA
agcttcaccttcagtgatgag

SEQ ID NO: 44 - DNA, 22 bp, synthetic construct
  misc_feature 1..22: note = Rgcc (NM_014059) gene-Reverse (5-3) primer sequence
  source 1..22: mol_type = other DNA
gtgtcacctaatttggctttcc

SEQ ID NO: 45 - DNA, 20 bp, synthetic construct
  misc_feature 1..20: note = Runx3 (NM_004350.3) gene-Forward (5-3) primer sequence
  source 1..20: mol_type = other DNA
ggttcaacgaccttcgcttc

SEQ ID NO: 46 - DNA, 19 bp, synthetic construct
  misc_feature 1..19: note = Runx3 (NM_004350.3) gene-Reverse (5-3) primer sequence
  source 1..19: mol_type = other DNA
cacggtcaccttgatggct

SEQ ID NO: 47 - DNA, 24 bp, synthetic construct
  misc_feature 1..24: note = Spi1 (NM_003120.3) gene-Forward (5-3) primer sequence
  source 1..24: mol_type = other DNA
catagcgatcactactgggatttc

SEQ ID NO: 48 - DNA, 22 bp, synthetic construct
  misc_feature 1..22: note = Spi1 (NM_003120.3) gene-Reverse (5-3) primer sequence
  source 1..22: mol_type = other DNA
ggttctcagggaagttctcaaa

SEQ ID NO: 49 - DNA, 20 bp, synthetic construct
  misc_feature 1..20: note = Stk11 (NG_007460) gene-Forward (5-3) primer sequence
  source 1..20: mol_type = other DNA
gccaacgtcaagaaggagat

SEQ ID NO: 50 - DNA, 23 bp, synthetic construct
  misc_feature 1..23: note = Stk11 (NG_007460) gene-Reverse (5-3) primer sequence
  source 1..23: mol_type = other DNA
cgcagtactccatcaccatatac

SEQ ID NO: 51 - DNA, 23 bp, synthetic construct
  misc_feature 1..23: note = Trerf1 (NM_001391983.1) gene-Forward (5-3) primer sequence
  source 1..23: mol_type = other DNA
gacaagtggacatccctagaaag

SEQ ID NO: 52 - DNA, 22 bp, synthetic construct
  misc_feature 1..22: note = Trerf1 (NM_001391983.1) gene-Reverse (5-3) primer sequence
  source 1..22: mol_type = other DNA
tgtcttggacttcaccatcttc

SEQ ID NO: 53 - DNA, 22 bp, synthetic construct
  misc_feature 1..22: note = Zeb1 (NG_017048.2) gene-Forward (5-3) primer sequence
  source 1..22: mol_type = other DNA
cttacggattcacagtggagag

SEQ ID NO: 54 - DNA, 22 bp, synthetic construct
  misc_feature 1..22: note = Zeb1 (NG_017048.2) gene-Reverse (5-3) primer sequence
  source 1..22: mol_type = other DNA
gtgagctataggagccagaatg

SEQ ID NO: 55 - DNA, 24 bp, synthetic construct
  misc_feature 1..24: note = Cdk14-ChIP (NM_001287135.2) gene-Forward (5-3) primer sequence
  source 1..24: mol_type = other DNA
gagatcatggtgttaagcaacaaa

SEQ ID NO: 56 - DNA, 19 bp, synthetic construct
  misc_feature 1..19: note = Cdk14-ChIP (NM_001287135.2) gene-Reverse (5-3) primer sequence
  source 1..19: mol_type = other DNA
acacacacacacacacaca

SEQ ID NO: 57 - DNA, 21 bp, synthetic construct
  misc_feature 1..21: note = Cyrib-ChIP (NM_016623.5) gene-Forward (5-3) primer sequence
  source 1..21: mol_type = other DNA
ggagaagtgaaaggagggttt

SEQ ID NO: 58 - DNA, 23 bp, synthetic construct
  misc_feature 1..23: note = Cyrib-ChIP (NM_016623.5) gene-Reverse (5-3) primer sequence
  source 1..23: mol_type = other DNA
tctctctctctctctctctctct

SEQ ID NO: 59 - DNA, 25 bp, synthetic construct
  misc_feature 1..25: note = Kif16b-ChIP (NG_028043) gene-Forward (5-3) primer sequence
  source 1..25: mol_type = other DNA
aacctcccttctgataaatagttga

SEQ ID NO: 60 - DNA, 23 bp, synthetic construct
  misc_feature 1..23: note = Kif16b-ChIP (NG_028043) gene-Reverse (5-3) primer sequence
  source 1..23: mol_type = other DNA
gtgctaccaaagaagatgaatgg

SEQ ID NO: 61 - DNA, 22 bp, synthetic construct
  misc_feature 1..22: note = Mad2l1-ChIP (NM_002358.4) gene-Forward (5-3) primer sequence
  source 1..22: mol_type = other DNA
gcctacatgctatctccattgt

SEQ ID NO: 62 - DNA, 23 bp, synthetic construct
  misc_feature 1..23: note = Mad2l1-ChIP (NM_002358.4) gene-Reverse (5-3) primer sequence
  source 1..23: mol_type = other DNA
ctctctctctctgtctgtcttct

SEQ ID NO: 63 - DNA, 26 bp, synthetic construct
  misc_feature 1..26: note = Nostrin-ChIP (NM_001039724.4) gene-Forward (5-3) primersequence
  source 1..26: mol_type = other DNA
ctctctaaataccagtcttccttctt

SEQ ID NO: 64 - DNA, 21 bp, synthetic construct
  misc_feature 1..21: note = Nostrin-ChIP (NM_001039724.4) gene - Reverse (5-3) primersequence
  source 1..21: mol_type = other DNA
ggaggaaagaaagggaaagga

SEQ ID NO: 65 - DNA, 21 bp, synthetic construct
  misc_feature 1..21: note = Plau-ChIP (NG_011904) gene-Forward (5-3) primer sequence
  source 1..21: mol_type = other DNA
cagtctgcccatgcaattatc

SEQ ID NO: 66 - DNA, 23 bp, synthetic construct
  misc_feature 1..23: note = Plau-ChIP (NG_011904) gene-Reverse (5-3) primer sequence
  source 1..23: mol_type = other DNA
cagaaagagtgtgtgtatgtgtg

SEQ ID NO: 67 - DNA, 22 bp, synthetic construct
  misc_feature 1..22: note = Trerf1-ChIP (NM_001391983.1) gene-Forward (5-3) primer sequence
  source 1..22: mol_type = other DNA
taactggcattctgggtaatgg

SEQ ID NO: 68 - DNA, 22 bp, synthetic construct
  misc_feature 1..22: note = Trerf1-ChIP (NM_001391983.1) gene-Reverse (5-3) primer sequence
  source 1..22: mol_type = other DNA
cacctcaggatttgtggtagtg

SEQ ID NO: 69 - DNA, 26 bp, synthetic construct
  misc_feature 1..26: note = Zeb1-ChIP (NG_017048.2) gene-Forward (5-3) primer sequence
  source 1..26: mol_type = other DNA
ctaaggtttctcctactgagaaagaa

SEQ ID NO: 70 - DNA, 21 bp, synthetic construct
  misc_feature 1..21: note = Zeb1-ChIP (NG_017048.2) gene-Reverse (5-3) primer sequence
  source 1..21: mol_type = other DNA
gccttgcttacaatggaatgg

SEQ ID NO: 71 - DNA, 22 bp, synthetic construct
  misc_feature 1..22: note = Id2 intron +1.3 kb (NM_002166)-geneForward (5-3) primersequence
  source 1..22: mol_type = other DNA
gaagccacagtttgagcattta

SEQ ID NO: 72 - DNA, 20 bp, synthetic construct
  misc_feature 1..20: note = Id2 intron +1.3 kb (NM_002166) gene-Reverse (5-3) primersequence
  source 1..20: mol_type = other DNA
gagactgggtttgggaagtt

SEQ ID NO: 73 - DNA, 20 bp, synthetic construct
  misc_feature 1..20: note = Runx3 intron kb (NM_004350.3) gene-Forward (5-3) primersequence
  source 1..20: mol_type = other DNA
tgaagcccagagactcagat

SEQ ID NO: 74 - DNA, 20 bp, synthetic construct
  misc_feature 1..20: note = Runx3 intron kb (NM_004350.3) gene-Reverse (5-3) primersequence
  source 1..20: mol_type = other DNA
taccagaggacccacattct

SEQ ID NO: 75 - DNA, 21 bp, synthetic construct
  misc_feature 1..21: note = Runx3 intron ±kb (NM_004350.3) gene-Forward (5-3) primersequence
  source 1..21: mol_type = other DNA
cacagctcaagcagggtatag

SEQ ID NO: 76 - DNA, 20 bp, synthetic construct
  misc_feature 1..20: note = Runx3 intron ±kb (NM_004350.3) gene-Reverse (5-3) primersequence
  source 1..20: mol_type = other DNA
ggagaccacagaggacttgt